(19) United States
(12) Patent Application Publication
(10) Pub. No.: US 2009/0318546 A1
(43) Pub. Date: Dec. 24, 2009

(54) Chromen-2-One Derivatives

(75) Inventors: Rolf Baenteli, Basel (CH); Nigel Graham Cooke, Oberwill (CH); Sven Weiler, Lorrach (DE); Frederic Zecri, Bartenheim (FR)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVARTIS AG, Basel (CH)

(21) Appl. No.: 12/296,317

(22) PCT Filed: Apr. 10, 2007
(86) PCT No.: PCT/EP2007/003184

§ 371 Date: Oct. 7, 2008

(30) Foreign Application Priority Data
Apr. 12, 2006 (GB) 0607389.4

(51) Int. Cl. A61K 31/37; C07D 311/04; A61P 37/02

(52) U.S. Cl. 514/457; 549/399

(57) ABSTRACT

A compound of formula (I)

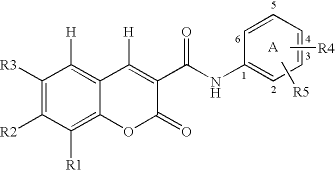

wherein R₁, R₂, R₃, R₄, R₅, and ring A are as defined in the specification, processes for their production, their uses, in particular in transplantation, and pharmaceutical compositions containing them.

The present invention relates to chromen-2-one derivatives, processes for their production, their use as pharmaceuticals and to pharmaceutical compositions comprising them. More particularly the present invention provides in a first aspect a compound of formula I

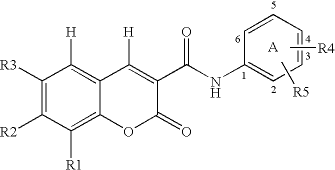

wherein

each of R₁ and R₂, independently, is selected from the group consisting of hydrogen; halogen; nitro; unsubstituted C₁₋₈alkyl; C₁₋₈alkyl substituted e.g. by aryl, C₃₋₆cycloalkyl, or C₁₋₈alkoxy; optionally substituted haloC₁₋₈alkyl; optionally substituted C₁₋₈alkyl-carbonyl; optionally substituted C₁₋₈alkenyl; unsubstituted C₁₋₈alkoxy; C₁₋₈alkoxy substituted e.g. by C₁₋₈alkoxy, C₃₋₈cycloalkyl, aryl, heterocyclic residue (e.g. heteroaryl, or heteroC₃₋₈cycloalkyl); C₁₋₈alkynyl; optionally substituted haloC₁₋₈alkoxy; unsubstituted C₃₋₆cycloalkyl; C₃₋₆cycloalkyl substituted e.g. by C₁₋₈alkyl; optionally substituted C₃₋₈cycloalkyl-oxy; heterocyclic residue; aryl optionally substituted e.g. by alkyl;

or R₁ and R₂ form together an optionally substituted C₃₋₈cycloalkyl or a heterocyclic residue;

R₃ is hydrogen; halogen; optionally substituted C₁₋₈alkyl (e.g. substituted by one or more C₁₋₈alkyl); optionally subtituted C₁₋₈alkoxy (e.g. substituted by C₁₋₈alkyl), optionally substituted haloC,-alkoxy (e.g. OCF₃); C₁₋₈alkenyl; preferably R3 is hydrogen; halogen; optionally substituted C₁₋₈alkyl; optionally subtituted C₁₋₈alkoxy; haloC₁₋₈alkoxy.

R₄ is of formula C₁₋₂alkyl-NR_{c}R_{d} wherein
the C₁₋₂alkyl is unsubstituted or subsbtituted by one or more substituents selected from hydroxyl; carboxyl; C₁₋₈alkyl (optionally substituted e.g. by hydroxyl, or carboxyl); C₃₋₆cycloalkyl optionally substituted by C₁₋₆alkyl; mono(C₁₋₆alkyl)carbamoyl; and di(C₁₋₆alkyl)₂carbamoyl;

or the C₁₋₂alkyl is substituted by two alkyl residues on the same C atom wherein, optionally, the two alkyl residues form together with the C atom to which they are bound a C₃₋₈cycloalkyl; preferably is substituted by two alkyl residues on the same C atom;
each of R_{c} and R_{d}, independently, is selected from the group consisting of hydrogen; unsubstituted C₁₋₈alkyl; C₁₋₈alkyl substituted e.g. by hydroxyl, carboxyl, C₁₋₈alkyl, C₁₋₈alkoxy, aryl, C₁₋₆alkoxycarbonyl, mono(C₁₋₆alkyl)carbamoyl, or di(C₁₋₆alkyl)₂carbamoyl); haloC₁₋₈alkyl; C₃₋₆cycloalkyl; C₁₋₆alkylcarbonyl; C₁₋₆alkoxycarbonyl; and C₁₋₆alkyne; or
R_{c} and R_{d} form together with the nitrogen atom to which they are bound an optionally substituted heterocyclic residue;

preferably R₄ is of formula Ia, Ib or Ic

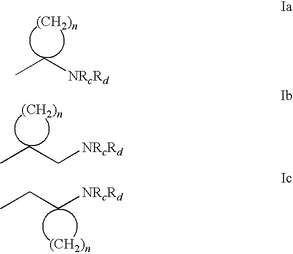

wherein n is 2 to 7, preferably 2 to 4, more preferably 2; and R_{c} and R_{d} are as hereainabove defined;

R₄ is in position 3 or 4, preferably 4;

R₅ is hydrogen; hydroxyl; halogen; haloC₁₋₈alkyl; optionally substituted C₁₋₈alkyl (e.g. unsubstituted C₁₋₈alkyl ); C₁₋₈alkoxy; or haloC₁₋₈alkoxy; preferably R₅ is hydrogen;
and R₅ is in position 2 (ortho) or 3 (meta), preferably 2;

or R₄ and R₅ are in position 4 and 3, respectively, and form together a heterocyclic residue;

ring A comprises no heteroatom or one or two ring heteroatom; preferably at the positions 2 and 3, preferably one or two nitrogen atoms;

with the proviso that when R₁ is hydrogen, then R₂ is not hydrogen, and reciprocally when R₂ is hydrogen, then R₁ is not hydrogen;

or a physiologically hydrolysable derivative thereof, a salt, hydrate and/or solvate thereof.

When ring A comprises one or two heteroatom, R₅ is preferably hydrogen.

Halogen may be fluorine, chlorine or bromine, preferably fluorine or chlorine.

Alkyl or alkoxy as a group or present in a group may be straight or branched. Alkylene may be straight or branched.

Alkyl as a group or present in a group may be substituted, e.g. by carboxyl, for example for R₄, R_{c}, R_{d}; hydroxyl, for example for R₄, R_{c}, R_{d}; alkoxy, for example for R₁, R₂, R_{c}, R_{d}; aryl, for example for R₁, R₂, R_{c}, R_{d}; aryl, for example for R₁, R₂; C₃₋₈cycloalkyl, for example for R₁, R₂, R₄; alkxycarbonyl, for example for R_{c}, R_{d}; mono(alkyl)carbamoyl or di(alkyl)₂carbamoyl, for example for R₄, R_{c}, R_{d}.

Alkoxy as a group or present in a group may be substituted, e.g. by hydroxyl, for example for R₁, R₂, R₃, R₅; carboxyl, for example for R₁, R₂, R₃; alkyl, for example for R₁, R₂, R₃, R₅; alkoxy, for example for R₁, R₂; heterocyclic residue, for example for R₁, R₂; C₃₋₈cycloalkyl, for example for R₁, R₂; heterocyclic residue (e.g. C₃₋₈cycloalkyl) for example for R₁, R₂; R₃; aryl, for example for R₁, R₂; heteroaryl, for example for R₁, R₂; or C₁₋₈alkynyl, for example for R₁, R₂.

When alkyl or alkoxy is substituted by hydroxyl, the hydroxyl group is preferably at the terminal position of the alkyl or alkoxy.

Alkenyl may be substituted e.g. by alkyl.

As herein defined haloalkyl and haloalkoxy refers to alkyl and alkoxy, respectively, either as a group or present in a group, which is substituted by 1 to 5 halogen, e.g. CF₃—, CHF₂—, CH₂F— or CF₃—CH₂—O—, CHF₂—CH₂—O—, CH₂F—CH₂—O—.

Haloalkyl and haloalkoxy may be substituted e.g. by alkyl, hydroxyl. Preferably haloalkyl and haloalkoxy are unsubstituted.

Any aryl may be phenyl or naphthyl, preferably phenyl.

Aryl may be substituted e.g. by alkyl, for example for R₁ or R₂.

By C₃₋₈cycloalkyl, as a group or present in a group, e.g. as R₁, R₂, as formed by R₁ and R₂, or as formed by the two alkyl residues bound on the same C atom of the C₁₋₂alkyl of R₄, is meant a three to eight, preferably five to seven, even more preferably three to five, membered non aromatic ring, comprising no heteroatom.

By heteroC₃₋₈cycloalkyl, as a group or present in a group, e.g. as R₁, R₂, is meant a three to eight, preferably five to seven, membered non aromatic ring, comprising 1 or 2 heteroatoms, preferably selected from N, O and S.

As hereinabove defined, C₁₋₂alkyl substituted by two alkyl residues on the same C atom wherein the two alkyl residues form together with the C atom to which they are bound a C₃₋₈cycloalkyl means any of 1-amino-C₃₋₈cycloalkyl, 1-aminomethyl-C₃₋₈cycloalkyl and 1-amino-C₃₋₈cycloalkylmethyl.

Cycloalkyl and cycloalkyl-oxy, as a group or present in a group, may be substituted e.g. by alkyl or halogen, for example for R₁, R₂, or R₄. Preferably cycloalkyl and cycloalkyl-oxy are unsubstituted.

By heterocyclic residue as R₁ or R₂, or formed respectively by R₁ and R₂, NR_{c}R_{d}, or R₄ and R₅, is meant a three to eight, preferably five to eight, membered saturated, unsaturated or aromatic heterocyclic ring comprising 1 or 2 heteroatoms, preferably selected from N, O and S, and optionally fused to another five to eight, membered saturated, unsaturated or aromatic heterocyclic ring. The heterocyclic residue is optionally substituted.

In case of the heterocyclic residue which may be formed by R_{c} and R_{d}, by heterocyclic residue it is also meant the N-oxide thereof.

When the heterocyclic residue is substituted, this may be on one or more ring carbon atoms and/or on a ring nitrogen atom when present. Examples of a substituent on a ring carbon atom include e.g. halogen, C₁₋₄alkyl, carbonyl, carboxyl, or hydroxyl, for example when the heterocyclic residue is formed by R_{c} and R_{d} or imino for example when the heterocyclic residue is formed by R₄ and R₅. Examples of a substituent on a ring heteroatom may include e.g. C₁₋₄alkyl, for example when the heterocyclic residue is formed by R_{c} and R_{d}, N-oxide

The following significances are preferred independently, collectively or in any combination or sub-combination:
1. each of R₁ and R₂, independently, is selected from the group consisting of hydrogen; optionally substituted C₁₋₈alkyl (e.g. C₁₋₈alkyl unsubstituted or substituted by aryl, C₃₋₆cycloalkyl, or C₁₋₈alkoxy); optionally substituted haloC₁₋₈alkyl; optionally substituted C₁₋₈alkoxy (e.g. C₁₋₈alkoxy unsubstituted or substituted by C₁₋₈alkoxy, C₃₋₈cycloalkyl, aryl, heterocyclic residue); optionally substituted haloC₁₋₈alkoxy; with the provido that R₁ and R₂ are not both hydrogen; with the proviso that R₁ and R₂ are not both hydrogen;
2. each of R₁ and R₂, independently, is selected from the group consisting of hydrogen; optionally substituted C₁₋₈alkyl (e.g. C₁₋₈alkyl unsubstituted or substituted by C₁₋₈alkoxy or aryl, preferably alkoxy); haloC₁₋₈alkyl; optionally substituted C₁₋₈alkoxy (e.g. C₁₋₈alkoxy substituted by C₁₋₈alkoxy); haloC₁₋₈alkoxy; with the proviso that R₁ and R₂ are not both hydrogen;
3. each of R₁ and R₂, independently, is selected from the group consisting of hydrogen; C₁₋₈alkyl; haloC₁₋₈alkyl; C₁₋₈alkoxy; haloC₁₋₈alkoxy; and R₁ and R₂ are not both hydrogen;
4. R₁ is hydrogen; C₁₋₈alkyl; haloC₁₋₈alkyl; C₁₋₈alkoxy; or haloC₁₋₈alkoxy, and R₂ is hydrogen; optionally substituted C₁₋₈alkoxy; or haloC₁₋₈alkoxy; with the proviso that R₁ and R₂ are not both hydrogen;
5. R₁ is hydrogen, C₁₋₈alkyl; C₁₋₈alkyl substituted by C₁₋₈alkoxy or aryl; haloC₁₋₈alkyl; C₁₋₈alkoxy; C₁₋₈alkoxy substituted by C₁₋₈alkoxy; or haloC₁₋₈alkoxy;
6. R₁ is hydrogen; C₁₋₈alkyl; haloC₁₋₈alkyl; C₁₋₈alkoxy; or haloC₁₋₈alkoxy;
7. R₂ is hydrogen; C₁₋₈alkyl; C₁₋₈alkyl substituted by C₁₋₈alkoxy or aryl; haloC₁₋₈alkyl; C₁₋₈alkoxy; C₁₋₈alkoxy substituted by C₁₋₈alkoxy; haloC₁₋₈alkoxy; or C₃₋₆cycloalkyl;
8. R₂ is hydrogen; C₁₋₈alkyl; C₁₋₈alkyl substituted by C₁₋₈alkoxy or aryl; haloC₁₋₈alkyl; C₁₋₈alkoxy; C₁₋₈alkoxy substituted by C₁₋₈alkoxy; or haloC₁₋₈alkoxy;
9. R₂ is hydrogen; C₁₋₈alkyl; haloC₁₋₈alkyl; C₁₋₈alkoxy; or haloC₁₋₈alkoxy;
10. R₂ is hydrogen; optionally substituted C₁₋₈alkoxy; or haloC₁₋₈alkoxy;
11. R₃ is hydrogen; halogen; C₁₋₈alkyl; haloC₁₋₈alkyl; C₁₋₈alkoxy; or haloC₁₋₈alkoxy; preferably R₃ is hydrogen;
12. R₅ is hydrogen; halogen; optionally substituted C₁₋₈alkyl (preferably unsubstituted C₁₋₈alkyl); haloC₁₋₈alkyl; C₁₋₈alkoxy; or haloC₁₋₈alkoxy; preferably R₅ is hydrogen;
13. each of R₁ and R₂, independently, is selected from the group consisting of hydrogen; optionally substituted C₁₋₈alkyl (e.g. C₁₋₈alkyl unsubstituted or substituted by C₁₋₈alkoxy or aryl; e.g. unsubstituted C₁₋₈alkyl); haloC₁₋₈alkyl; optionally substituted C₁₋₈alkoxy (e.g. C₁₋₈alkoxy unsubstituted or substituted by C₁₋₈alkoxy; e.g. unsubstituted C₁₋₈alkoxy); and haloC₁₋₈alkoxy; and R₃ is hydrogen; halogen; C₁₋₈alkyl; haloC_{1,8}alkyl; C₁₋₈alkoxy; or haloC₁₋₈alkoxy; preferably R₃ is hydrogen; with the proviso that R₁ and R₂ are not both hydrogen;
14. each of R₁ and R₂, independently, is selected from the group consisting of hydrogen; optionally substituted C₁₋₈alkyl (e.g. C₁₋₈alkyl unsubstituted or substituted by C₁₋₈alkoxy or aryl; e.g. unsubstituted C₁₋₈alkyl); haloC₁₋₈alkyl; optionally substituted C₁₋₈alkoxy (e.g. C₁₋₈alkoxy unsubstituted or substituted by C₁₋₈alkoxy; e.g. unsubstituted C₁₋₈alkoxy); and haloC₁₋₈alkoxy; and R₅ is hydrogen; halogen; optionally substituted C₁₋₈alkyl (preferably unsubstituted C₁₋₈alkyl); haloC₁₋₈alkyl; C₁₋₈alkoxy; or haloC₁₋₈alkoxy; preferably R₅ is hydrogen;
15. each of R₁ and R₂, independently, is selected from the group consisting of hydrogen; optionally substituted C₁₋₈alkyl (e.g. C₁₋₈alkyl unsubstituted or substituted by C₁₋₈alkoxy or aryl; e.g. unsubstituted C₁₋₈alkyl); haloC₁₋₈alkyl; optionally substituted C₁₋₈alkoxy (e.g. C₁₋₈alkoxy unsubstituted or substituted by C₁₋₈alkoxy; e.g. unsubstituted C₁₋₈alkoxy); and haloC₁₋₈alkoxy; R₅ is hydrogen; halogen; optionally substituted C₁₋₈alkyl (preferably unsubstituted C₁₋₈alkyl); haloC₁₋₈alkyl; C₁₋₈alkoxy; or haloC₁₋₈alkoxy, preferably R₅ is hydrogen, and R₃ is hydrogen; halogen; C₁₋₈alkyl; haloC₁₋₈alkyl; C₁₋₈alkoxy; or haloC₁₋₈alkoxy; preferably R₃ and R₅ are both hydrogen;
16. R₄ is of formula C₁₋₂alkyl-NR_{c}R_{d} wherein the C₁₋₂alkyl is optionally substituted or is substituted by two alkyl residues on the same C atom wherein the two alkyl residues optionally form a C₃₋₈cycloalkyl; preferably R₄ is of formula C₁₋₂alkyl-NR_{c}R_{d} wherein the C₁₋₂alkyl is substituted by two alkyl residues on the same C atom or R₄ is of formula Ia, Ib or Ic;
17. each of R_{c} and R_{d}, independently, is hydrogen; optionally substituted C₁₋₈alkyl (e.g. C₁₋₈alkyl unsubstituted or substituted by hydroxyl); or haloC₁₋₈alkyl; or R_{c} and R_{d} form together with the nitrogen atom to which they are bound a heterocyclic residue;
18. R₄ is of formula C₁₋₂alkyl-NR_{c}R_{d} wherein the C₁₋₂alkyl is optionally substituted or is substituted by two alkyl residues on the same C atom wherein the two alkyl residues optionally form a C₃₋₈cycloalkyl (e.g. R₄ is of formula Ia, Ib or Ic); and each of R_{c} and R_{d}, independently, is hydrogen; optionally substituted C₁₋₈alkyl (e.g. C₁₋₈alkyl unsubstituted or substituted by hydroxyl); or haloC₁₋₈alkyl; or R_{c} and R_{d} form together with the nitrogen atom to which they are bound a heterocyclic residue;
19. R₃ is hydrogen; halogen; C₁₋₈alkyl; haloC₁₋₈alkyl; C₁₋₈alkoxy; or haloC₁₋₈alkoxy; preferably R₃ is hydrogen; and R₄ is of formula C₁₋₂alkyl-NR_{c}R_{d} wherein the C₁₋₂alkyl is optionally substituted or is substituted by two alkyl residues on the same C atom wherein the two alkyl residues optionally form a C₃₋₈cycloalkyl (e.g. R₄ is of formula Ia, Ib or Ic);
20. R₃ is hydrogen; halogen; C₁₋₈alkyl; haloC₁₋₈alkyl; C₁₋₈alkoxy; or haloC₁₋₈alkoxy; preferably R₃ is hydrogen; and each of R_{c} and R_{d}, independently, is hydrogen; optionally substituted C₁₋₈alkyl (e.g unsubstituted C₁₋₈alkyl or C₁₋₈alkyl substituted by hydroxyl); or haloC₁₋₈alkyl; or R_{c} and R_{d} form together with the nitrogen atom to which they are bound a heterocyclic residue;
21. each of R₁ and R₂, independently, is selected from the group consisting of hydrogen; optionally substituted C₁₋₈alkyl (e.g. unsubstituted C₁₋₈alkyl or C₁₋₈alkyl substituted by C₁₋₈alkoxy or aryl); haloC₁₋₈alkyl; optionally substituted C₁₋₈alkoxy (e.g. unsubstituted C₁₋₈alkoxy or C₁₋₈alkoxy substituted by C₁₋₈alkoxy); or haloC₁₋₈alkoxy; and R₁ and R₂ are not both hydrogen; R₃ is hydrogen; halogen; C₁₋₈alkyl; haloC₁₋₈alkyl; C₁₋₈alkoxy; or haloC₁₋₈alkoxy; preferably R₃ is hydrogen; and R₄ is of formula C₁₋₂alkyl-NR_{c}R_{d} wherein the C₁₋₂alkyl is optionally substituted or is substituted by two alkyl residues on the same C atom wherein the two alkyl residues optionally form a C₃₋₈cycloalkyl; preferably R₄ is of formula C₁₋₂alkyl-NR_{c}R_{d} wherein the C₁₋₂alkyl is substituted by two alkyl residues on the same C atom or R₄ is of formula Ia, Ib or Ic;
22. each of R₁ and R₂, independently, is selected from the group consisting of hydrogen; optionally substituted C₁₋₈alkyl (e.g. unsubstituted C₁₋₈alkyl or C₁₋₈alkyl substituted by C₁₋₈alkoxy or aryl); haloC₁₋₈alkyl; optionally substituted C₁₋₈alkoxy (e.g. unsubstituted C₁₋₈alkoxy or C₁₋₈alkoxy substituted by C₁₋₈alkoxy); or haloC₁₋₈alkoxy; and R₁ and R₂ are not both hydrogen; R₃ is hydrogen; halogen; C₁₋₈alkyl; haloC₁₋₈alkyl; C₁₋₈alkoxy; or haloC₁₋₈alkoxy; preferably R₃ is hydrogen; and each of R_{c} and R_{d}, independently, is hydrogen; optionally substituted C₁₋₈alkyl (e.g. unsubstituted C₁₋₈alkyl or C₁₋₈alkyl substituted by hydroxyl); or haloC₁₋₈alkyl; or R_{c} and R_{d} form together with the nitrogen atom to which they are bound a heterocyclic residue;
23. each of R₁ and R₂, independently, is selected from the group consisting of hydrogen; optionally substituted C₁₋₈alkyl (e.g. unsubstituted C₁₋₈alkyl or C₁₋₈alkyl substituted by C₁₋₈alkoxy or aryl); haloC₁₋₈alkyl; optionally substituted C₁₋₈alkoxy (e.g. unsubstituted C₁₋₈alkoxy or C₁₋₈alkoxy substituted by C₁₋₈alkoxy); or haloC₁₋₈alkoxy; and R₁ and R₂ are not both hydrogen; and R₄ is of formula C₁₋₂alkyl-NR_{c}R_{d} wherein the C₁₋₂alkyl is optionally substituted or is substituted by two alkyl residues on the same C atom wherein the two alkyl residues optionally form a C₃₋₈cycloalkyl (e.g. R₄ is of formula Ia, Ib or Ic);
24. each of R₁ and R₂, independently, is selected from the group consisting of hydrogen; optionally substituted C₁₋₈alkyl (e.g. unsubstituted C₁₋₈alkyl or C₁₋₈alkyl substituted by C₁₋₈alkoxy or aryl); haloC₁₋₈alkyl; optionally substituted C₁₋₈alkoxy (e.g. unsubstituted C₁₋₈alkoxy or C₁₋₈alkoxy substituted by C₁₋₈alkoxy); or haloC₁₋₈alkoxy; and R₁ and R₂ are not both hydrogen; and each of R_{c} and R_{d}, independently, is hydrogen; optionally substituted C₁₋₈alkyl (e.g. unsubstituted C₁₋₈alkyl or C₁₋₈alkyl substituted by hydroxyl); or haloC₁₋₈alkyl; or R_{c} and R_{d} form together with the nitrogen atom to which they are bound a heterocyclic residue;
25. R₄ is of formula C₁₋₂alkyl-NR_{c}R_{d} wherein the C, ₂alkyl is optionally substituted or is substituted by two alkyl residues on the same C atom wherein the two alkyl residues optionally form a C₃₋₈cycloalkyl (e.g. R₄ is of formula Ia, Ib or Ic); and R₅ is hydrogen; halogen; optionally substituted C₁₋₈alkyl (preferably unsubstituted C₁₋₈alkyl); haloC₁₋₈alkyl; C₁₋₈alkoxy; or haloC₁₋₈alkoxy; preferably R₅ is hydrogen;
26. each of R_{c} and R_{d}, independently, is hydrogen; optionally substituted C₁₋₈alkyl (e.g. unsubstituted C₁₋₈alkyl or C₁₋₈alkyl substituted by hydroxyl); haloC₁₋₈alkyl; or R_{c} and R_{d} form together with the nitrogen atom to which they are bound a heterocyclic residue; and R₅ is hydrogen; halogen; optionally substituted C₁₋₈alkyl (preferably unsubstituted C₁₋₈alkyl); haloC₁₋₈alkyl; C₁₋₈alkoxy; or haloC₁₋₈alkoxy; preferably R₅ is hydrogen;
27. R₅ is hydrogen; halogen; optionally substituted C₁₋₈alkyl (preferably unsubstituted C₁₋₈alkyl); haloC₁₋₈alkyl; C₁₋₈alkoxy; or haloC₁₋₈alkoxy; and R₃ is hydrogen; halogen; C₁₋₈alkyl; haloC₁₋₈alkyl; C₁₋₈alkoxy; or haloC₁₋₈alkoxy; preferably R₃ and R₅ are both hydrogen;
28. R₄is of formula C₁₋₂alkyl-NR_{c}R_{d} wherein the C₁₋₂alkyl is optionally substituted or is substituted by two alkyl residues on the same C atom wherein the two alkyl residues optionally form a C₃₋₈cycloalkyl (e.g. R₄is of formula Ia, Ib or Ic); R₅is hydrogen; halogen; optionally substituted C₁₋₈alkyl (preferably unsubstituted C₁₋₈alkyl); haloC₁₋₈alkyl; C₁₋₈alkoxy; or haloC₁₋₈alkoxy; and R₃is hydrogen; halogen; C₁₋₈alkyl; haloC₁₋₈alkyl; C₁₋₈alkoxy; or haloC₁₋₈alkoxy; preferably R₃ and R₅ are both hydrogen;
29. each of R_{c} and R_{d}, independently, is hydrogen; optionally substituted C₁₋₈alkyl (e.g. unsubstituted C₁₋₈alkyl or C₁₋₈alkyl substituted by hydroxyl); or haloC₁₋₈alkyl; or R_{c} and R_{d} form together with the nitrogen atom to which they are bound a heterocyclic residue; R₅ is hydrogen; halogen; optionally substituted C₁₋₈alkyl (preferably unsubstituted C₁₋₈alkyl); haloC₁₋₈alkyl; C₁₋₈alkoxy; or haloC₁₋₈alkoxy; and R₃is hydrogen; halogen; C₁₋₈alkyl; haloC₁₋₈alkyl; C₁₋₈alkoxy; or haloC₁₋₈alkoxy; preferably R₃ and R₅ are both hydrogen;
30. R₄ is of formula C₁₋₂alkyl-NR_{c}R_{d} wherein the C₁₋₂alkyl is optionally substituted or is substituted by two alkyl residues on the same C atom wherein the two alkyl residues optionally form a C₃₋₈cycloalkyl (e.g. R₄ is of formula Ia, Ib or Ic); each of R_{c} and R_{d}, independently, is hydrogen; optionally substituted C₁₋₈alkyl (e.g. unsubstituted C₁₋₈alkyl or C₁₋₈alkyl substituted by hydroxyl);or haloC₁₋₈alkyl; or R_{c} and R_{d} form together with the nitrogen atom to which they are bound a heterocyclic residue; R₅ is hydrogen; halogen; optionally substituted C₁₋₈alkyl (preferably unsubstituted C₁₋₈alkyl); haloC₁₋₈alkyl; C₁₋₈alkoxy; or haloC₁₋₈alkoxy; and R₃ is hydrogen; halogen; C₁₋₈alkyl; haloC₁₋₈alkyl; C₁₋₈alkoxy; or haloC₁₋₈alkoxy; preferably R₃ and R₅ are both hydrogen;
31. each of R₁ and R₂, independently, is selected from the group consisting of hydrogen; optionally substituted C₁₋₈alkyl (e.g. unsubstituted C₁₋₈alkyl or C₁₋₈alkyl substituted by C₁₋₈alkoxy or aryl); haloC₁₋₈alkyl; optionally substituted C₁₋₈alkoxy (e.g. unsubstituted C₁₋₈alkoxy or C₁₋₈alkoxy substituted by C₁₋₈alkoxy); or haloC₁₋₈alkoxy; and R₁ and R₂ are not both hydrogen; R₄ is of formula C₁₋₂alkyl-NR_{c}R_{d} wherein the C₁₋₂alkyl is optionally substituted or is substituted by two alkyl residues on the same C atom wherein the two alkyl residues optionally form a C₃₋₈cycloalkyl (e.g. R₄ is of formula Ia, Ib or Ic); each of R_{c} and R_{d}, independently, is hydrogen; optionally substituted C₁₋₈alkyl (e.g. unsubstituted C₁₋₈alkyl or C₁₋₈alkyl substituted by hydroxyl); or haloC₁₋₈alkyl; or R_{c} and R_{d} form together with the nitrogen atom to which they are bound a heterocyclic residue; R₅ is hydrogen; halogen; optionally substituted C₁₈alkyl (preferably unsubstituted C₁₋₈alkyl); haloC₁₋₈alkyl; C₁₋₈alkoxy; or haloC₁₋₈alkoxy; and R₃ is hydrogen; halogen; C₁₋₈alkyl; haloC₁₋₈alkyl; C₁₋₈alkoxy; or haloC₁₋₈alkoxy; preferably R₃ and R₅ are both hydrogen;
32. R₄ is of formula C₁₋₂alkyl-NR_{c}R_{d} wherein the C₁₋₂alkyl is optionally substituted or is substituted by two alkyl residues on the same C atom wherein the two alkyl residues optionally form a C₃₋₈cycloalkyl; each of R_{c} and R_{d}, independently, is hydrogen; optionally substituted C₁₋₈alkyl (e.g. unsubstituted C₁₋₈alkyl or C₁₋₈alkyl substituted by hydroxyl); or haloC₁₋₈alkyl; or R_{c} and R_{d} form together with the nitrogen atom to which they are bound a heterocyclic residue; and R₃ is hydrogen; halogen; C₁₋₈alkyl; haloC₁₋₈alkyl; C₁₋₈alkoxy; or haloC₁₋₈alkoxy; preferably R₃ is hydrogen;
33. R₄ is of formula C₁₋₂alkyl-NR_{c}R_{d} wherein the C₁₋₂alkyl is optionally substituted or is substituted by two alkyl residues on the same C atom wherein the two alkyl residues optionally form a C₃₋₈cycloalkyl (e.g. R₄ is of formula Ia, Ib or Ic); each of R_{c} and R_{d}, independently, is hydrogen; optionally substituted C₁₋₈alkyl (e.g. unsubstituted C₁₋₈alkyl or C₁₋₈alkyl substituted by hydroxyl);or haloC₁₋₈alkyl; or R_{c} and R_{d} form together with the nitrogen atom to which they are bound a heterocyclic residue; and R₅ is hydrogen; halogen; optionally substituted C₁₋₈alkyl; haloC₁₋₈alkyl; C₁₋₈alkoxy; or haloC₁₋₈alkoxy; preferably R₅ is hydrogen;
34. R₄ is in position 4;
35. Ring A comprises no heteroatom;
36. Ring A comprises 1 or 2 heteroatom, preferably one or two N atoms;
37. Ring A comprises 1 or 2 heteroatom, preferably one or two N atoms, on positions 2 and/or 3;
38. each of R₁ and R₂, independently, is selected from the group consisting of hydrogen; C₁₋₈alkyl; haloC₁₋₈alkyl; C₁₋₈alkoxy; haloC₁₋₈alkoxy; and R₁ and R₂ are not both hydrogen; R₄ is of formula C₁₋₂alkyl-NR_{c}R_{d} wherein the C₁₋₂alkyl is optionally substituted or is substituted by two alkyl residues on the same C atom wherein the two alkyl residues optionally form a C₃₋₈cycloalkyl (e.g. R₄ is of formula C₁₋₂alkyl-NR_{c}R_{d} wherein the C₁₋₂alkyl is substituted by two alkyl residues on the same C atom or R₄ is of formula Ia, Ib or Ic); each of R_{c} and R_{d}, independently, is hydrogen; optionally substituted C₁₋₈alkyl (e.g. unsubstituted C₁₋₈alkyl or C₁₋₈alkyl substituted by hydroxyl); or haloC₁₋₈alkyl; or R_{c} and R_{d} form together with the nitrogen atom to which they are bound a heterocyclic residue; R₄ is in position 4; R₅ is hydrogen; halogen; optionally substituted C₁₋₈alkyl (preferably unsubstituted C₁₋₈alkyl); haloC₁₋₈alkyl; C₁₋₈alkoxy; or haloC₁₋₈alkoxy; and R₃ is hydrogen; halogen; C₁₋₈alkyl; haloC₁₋₈alkyl; C₁₋₈alkoxy; or haloC₁₋₈alkoxy; preferably R₃ and R₅ are both hydrogen; and ring A comprises no heteroatom.

The compounds of formula I may exist in free form or in salt form, e.g. addition salts with e.g. organic or inorganic acids, for example, hydrochloric acid or acetic acid, or salts obtainable when R5 is or comprises COOH, with a base, e.g. alkali salts such as sodium or potassium, or substituted or unsubstituted ammonium salts.

It will be appreciated that the compounds of formula I may exist in the form of optical isomers, racemates or diastereoisomers. It is to be understood that the present invention embraces all enantiomers and conformers and their mixtures. Similar considerations apply in relation to starting materials exhibiting asymmetric carbon atoms as mentioned above.

By a physiologically hydrolysable derivative of a compound of formula I is meant a compound which is hydrolysable under physiological conditions to yield a compound of formula I and a by-product which is itself physiologically acceptable, e.g. an ester which is hydrolyzed to yield a compound of formula I and a non-toxic alcohol at the desired dosage levels.

The present invention also includes processes for the production of a compound of formula I (scheme 1), which processes comprise

either reacting a compound of formula II, wherein R₁, R₂, and R₃ are as defined above with a compound of formula III, wherein R₅ and R₄ are as defined above (route A);

or transforming a compound of formula II via an intermediate IV into an intermediate of formula V, wherein R₁, R₂ and R₃ are as defined above, and reacting it with a compound of formula VI, wherein R₅ and R₄ are as defined above (route B).

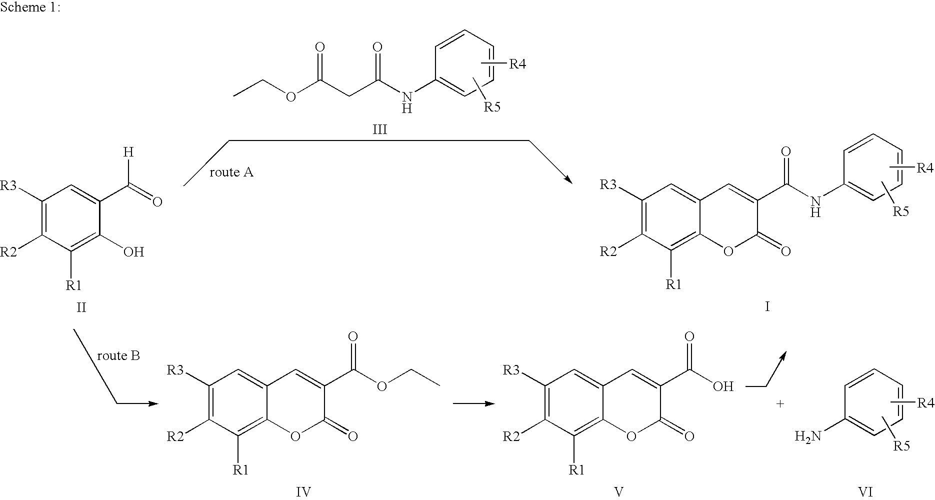

All reactions are performed in a solvent such as methanol, ethanol, tetrahydrofuran, toluene, dichloromethane, 1,2-dichloroethane, N-methyl pyrolidone, xylenes, ethyl acetate, diethyl ether, hexanes, cyclohexanes, dimethylformamide, acetone, dimethylsulfoxide, tert-butylmethyl ether. All compounds can be isolated using methods known to those skilled in the art (e.g. crystallization, silica gel chromatography, HPLC).

Following route A 2-hydroxy benzaldehydes of formula II can be condensated with the malonate derivatives of formula III in the presence of a suitable base (for example a secondary amine such as piperidine) in a suitable solvent.

Following route B 2-hydroxy benzaldehydes of formula II are condensated with diethyl malonate in the presence of a suitable base (for example a secondary amine such as piperidine) in a suitable solvent to give 2-oxo-2H-chromene-3-carboxylic ester of formula IV. In case, R2 is equal to hydroxy, at this stage the hydroxyl group can be alkylated to give R2 equals alkoxy under basic conditions using an alkylhalide as electrophile in presence of a suitable base seach as triethyl amine, piperidine, sodium hydride, potassium carbonate or cesium carbonate in presence of a suitable solvent, or using Mitsunobu conditions with the corresponding alcohol in presence of triphenyl phosphine and DEAD reagent.

2-Oxo-2H-chromene-3-carboxylic esters of formula IV are then saponified in presence of lithium hydroxide or sodium hydroxide in a suitable solvent to give 2-oxo-2H-chromene-3-carboxylic acids of formula V.

Compounds of formula V are activated for amide bond formation with a reagent such as thionyl chloride or 1-(3-dimethylaminopropyl)-3-ethylcarbodiimide or 1,1′-carbonyldiimidazole or propanephosphonic anhydride in the presence of a suitable base such as triethyl amine, N,N-diisopropylethylamine or sodium bicarbonate in a suitable solvent and reacted with a compound of formula VI (aniline derivative) leading to the desired compound of formula I. If R5 or R4 contains a nitrogen functionality protecting group e.g. a carbamic acid tert-butyl ester function, deprotection is effected by reacting it with an acid such as hydrochloric acid or trifluoroacetic acid in a suitable solvent.

Methods to prepare 2-oxo-2H-chromene-3-carboxylic acid and compounds of formula I using routes A and B as well as other methods pertinent to the present invention are known to the one skilled in the art and have been reviewed in the literature (Horing, E. C. et al. (1955) organic synthesis, Coll. Vol. III, 165, Livingstone, R. (1977); Rodd's Chemistry of carbon compounds, Vol. IV, p 96, Staunton, J. (1979); Heterocyclic Chemistry (ed. P. G. Sammes), Vol. 4).

Insofar as the production of the starting materials is not particularly described, the compounds are either known or may be prepared analogously to methods known in the art or as disclosed hereinafter.

A convenient method to prepare non-commercial 2-hydroxy benzaldehyde compounds of formula II wherein R₁ is allyl or propyl is shown in scheme 2 (route C). 2-Hydroxy benzaldehydes of formula II wherein R₁ is H can be O-alkylated with an electrophile such as allylbromide in presence of a suitable base such as potassium carbonate or cesium carbonate in a suitable solvent to give compound of formula VII. Claisen rearrangement of compounds of formula VII under thermic conditions (oil bath or microwave heating) can be carried out neat or in a suitable solvent to obtain compounds of formula II wherein R₁ is allyl. Selective reduction of the double bond in presence of the aldehyde to give compounds of formula II wherein R₁ is propyl can be achieved under standard hydrogenation conditions using Raney Nickel as a catalyst in a suitable solvent.

Alternatively as shown in scheme 2 (route D) if R₂ is alkoxy, compounds of formula VIII are reacted with a strong base such as butyl lithium and an alkyl halide or an acyl halide to give compounds of formula IX, which are O-dealkylated by the action of an acid such as hydrochloric acid, hydrobromic acid or boron tribromide in a suitable solvent to give compounds of formula X (phenols). Compounds of formula X are converted into compounds of formula II wherein R₁ is alkyl, or —COalkyl, and R₂ is OH under Vilsmeier conditions using for example POCl₃ and N,N-dimethylformamide as a carbonyl source in a suitable solvent.

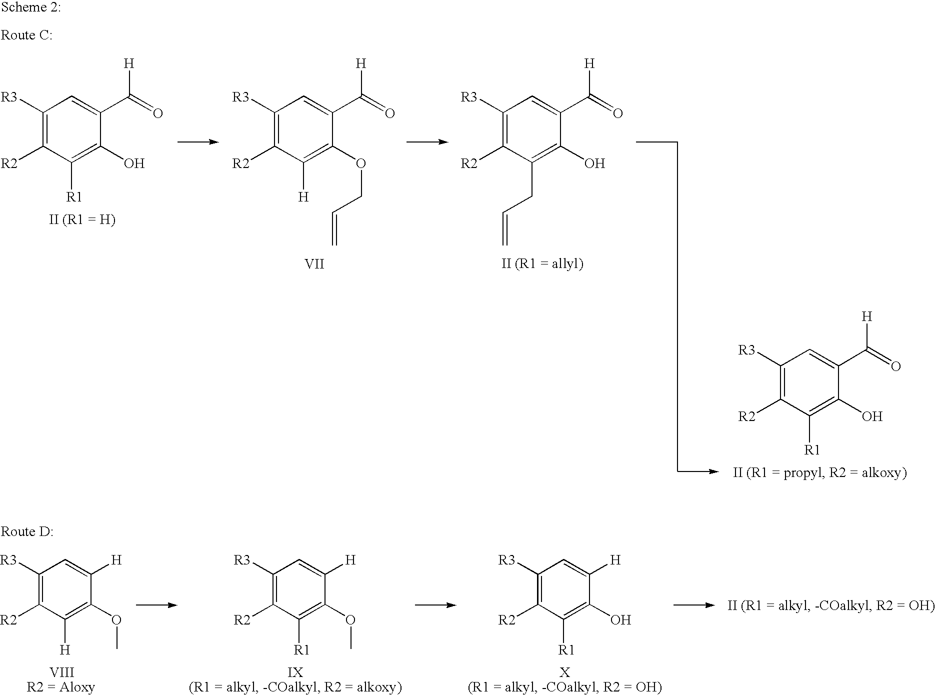

A convenient method to prepare non-commercial 2-hydroxy benzaldehyde intermediates of formula II wherein R₂ is hydroxy is shown in scheme 3. The synthesis of compounds of formula XI is reproduced according to a literature procedure (Synthetic Communications, 20(12), 1869-1876). Compounds of formula XI are converted into compounds of formula II wherein R₂ is hydroxy under Vilsmeier conditions using for example POCl₃ and N,N-dimethylformamide as a carbonyl source in a suitable solvent.

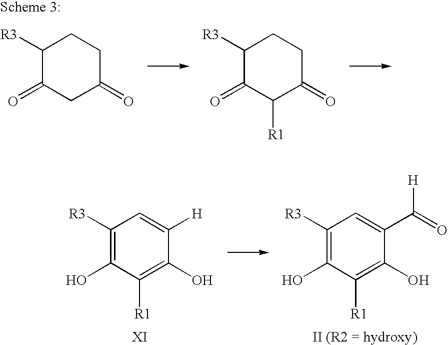

A convenient method to synthesize compound of formula III is shown in scheme 4. below. A compound of formula VI (aniline) is reacted under thermic condition with diethyl malonate as solvent. Alternatively monoethyl malonic acid can be activated with a reagent such as thionyl chloride or 1-(3-dimethylaminopropyl)-3-ethylcarbodiimide or 1,1′-carbonyldiimidazole and reacted with compound of formula VI in presence of a suitable base such as triethyl amine, N,N-diisopropylethylamine or sodium bicarbonate in a suitable solvent.

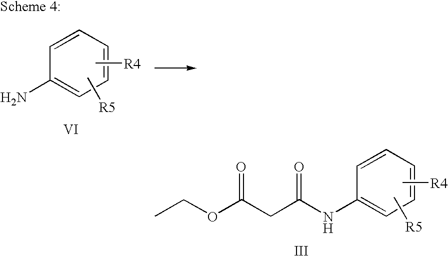

Aniline intermediates of formula VI can be purchased or the respective nitro compounds are purchased and reduced to the anilines of formula VI by the action of palladium on charcoal and hydrogen or palladium on charcoal with sodiumborohydride or tindichloride in a suitable solvent. Amino functions in R₄ and R₅ are protected as a tert-butoxycarbamate using BOC anhydride as an electrophile in presence of a suitable base such as triethyl amine, diethyl isopropyl amine in a suitable solvent.

Anilines of formula VI wherein R₄ is an optionally substituted aminomethyl group (CR′R′NR_{c}R_{d}) can be prepared by methods state in the art or one of the three routes E-G outlined in scheme 5.

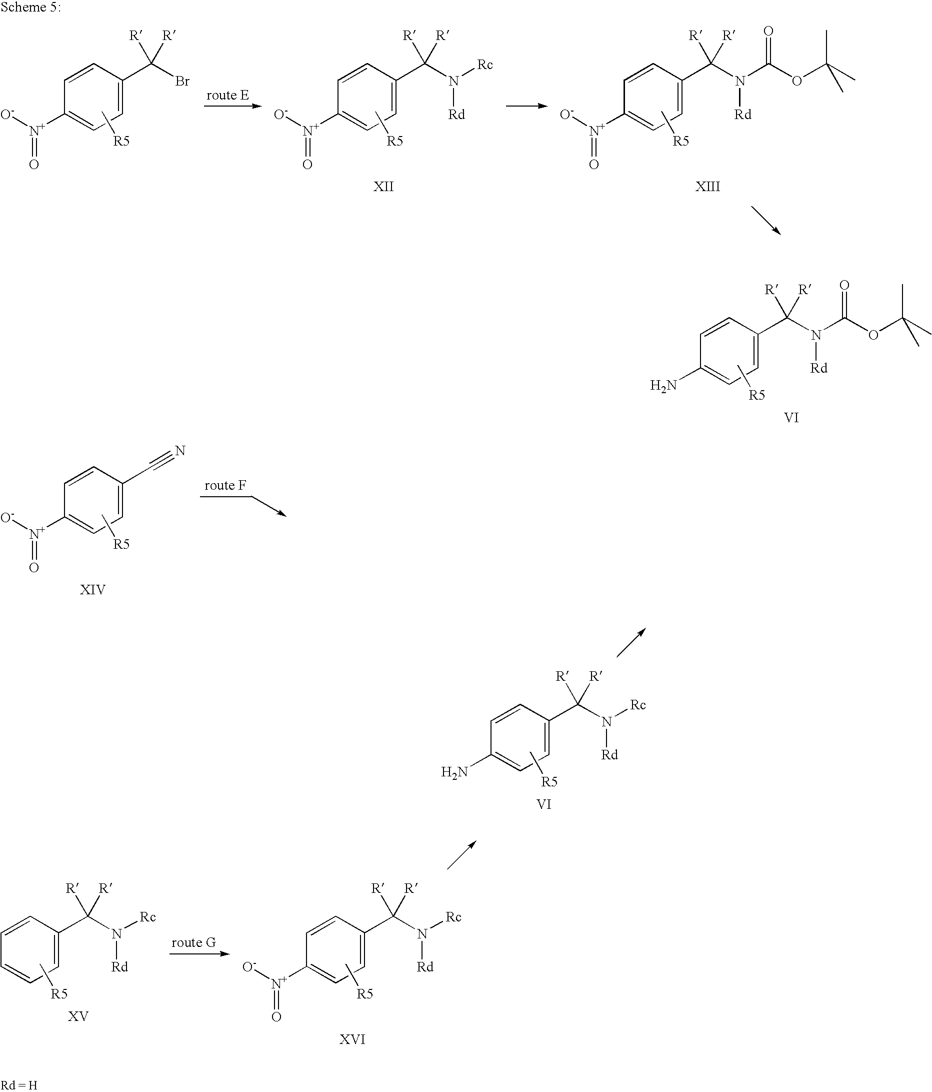

According to route E (scheme 5) 4-nitro benzyl bromide analogues can be converted to the benzyl amine intermediate of formula XII by nucleophilic displacement with the corresponding amine in presence of a suitable base such as triethyl amine, diethyl isopropyl amine in a suitable solvent.

When Rd and/or Rc is hydrogen, protection of the amine functionality of compound of formula XII can be carried out using BOC anhydride as electrophile in the presence of a suitable base such as triethyl amine, diethyl isopropyl amine in a suitable solvent to give compounds of formula XIII. Reduction of the nitro functionality in presence of a catalyst such as Pd on charcoal or Raney Nickel using hydrogen gas or sodium borohydride as hydrogen source in a suitable solvent gives intermediates of formula VI.

According to route F commercially available nitronitriles of formula XIV are reduced using Pd on charcoal or Raney nickel and hydrogen in a suitable solvent to give compounds of formula VI wherein R4 is CR′R′NR_{d}R_{c}). If Rc is hydrogen the amino function is protected using BOC anhydride as electrophile in presence of a suitable base such as triethyl amine, diethyl isopropyl amine in a suitable solvent to give compounds of formula VI wherein R4 is CR′R′NR_{d}BOC).

According to route G aniline intermediates of formula VI wherein R4 is CR′R′NR_{d}R_{c} can prepared from intermediates of formula XV using standard nitration conditions using nitric acid-sulfuric acid mixtures to give compounds of formula XVI which are reduced under standard reduction conditions in presence of a catalyst such as Pd on charcoal or Raney Nickel using hydrogen gas or sodium borohydride as hydrogen source in a suitable solvent. If R_{c} is hydrogen the amino function is protected using BOC anhydride as electrophile in presence of a suitable base such as triethyl amine, diethyl isopropyl amine in a suitable solvent.

The following examples are illustrative of the invention.

Concentration of solutions is carried out on a rotary evaporator under reduced pressure. Conventional flash chromatography is carried out on silica gel. Flash chromatography is also carried out using Biotage Flash Chromatography apparatus or Flashmaster instrument.

Abbreviations used are:

TBME=tert-butylmethyl ether

BOC=tert-butyloxy carbonyl

DMF=dimethylformamide

LiOH=lithium hydroxide

HCl=hydrochloric acid

THF=tetrahydrofuran

CH₂Cl₂=dichloromethane

RT=room temperature

NaOH=sodium hydroxide

Min=minute

### EXAMPLE 1

### 7-Methoxy-2-oxo-8-propyl-2H-chromene-3-carboxylic acid (4-aminomethyl-phenyl)-amide

| R₁ | R₂ | R₃ | R₄ | R₅ | (M + H)⁺ |
|---|---|---|---|---|---|
| propyl | methoxy | H | 4-CH2NH2 | H | (M + H—NH₃)⁺ = 350 |

### a) Preparation of 2-allyloxy4-methoxy-benzaldehyde

To a solution of 2-hydroxy-4-methoxy-benzaldehyde (5 g, 32.8 mmol) and allyl bromide (3.89 ml, 46 mmol) in acetone (50 ml) is added potassium carbonate (6.8 g, 49.3 mmol). The reaction mixture is then stirred under reflux for 3 hours. The reaction mixture is concentrated and partitioned between 200 ml of TBME and 150 ml of 1N NaOH and the layers were separated. The organic layer is washed with 150 ml of brine and 150 ml of water, dried and concentrated. 2-allyloxy-4-methoxy-benzaldehyde is isolated after purification using flash chromatography (eluent CH₂Cl₂/Hexanes 8/2).

### b) Preparation of 3-allyl-2-hydroxy-4-methoxy-benzaldehyde

A solution of 2-allyloxy-4-methoxy-benzaldehyde (5 g, 26 mmol) in NMP (10 ml) is microwave heated at 230° C. for 30 minutes. The reaction mixture is then poured into an ice/water (200 ml) mixture and TBME (200 ml) is added, the organic layer is separated and washed with 150 ml of brine and 150 ml of water, dried and concentrated. 3-Allyl-2-hydroxy-4-methoxy-benzaldehyde is isolated after purification using flash chromatography (eluent CH₂Cl₂/Hexanes 8/2).

### c) Preparation of 2-hydroxy-4-methoxy-3-propyl-benzaldehyde

To a solution of 3-allyl-2-hydroxy-4-methoxy-benzaldehyde (5 g, 26 mmol) in THF (25 ml) is added 10% wt Pt/C. The reaction mixture is then stirred at room temperature until 1 eq of hydrogen gas is consumed. The reaction mixture is then filtered over celite and concentrated. 2-hydroxy-4-methoxy-3-propyl-benzaldehyde is used without further purification.

### d) Preparation of 7-methoxy-2-oxo-8-propyl-2H-chromene-3-carboxylic acid ethyl ester

To a solution of 2-hydroxy-4-methoxy-3-propyl-benzaldehyde (15 g, 77.2 mmol) in ethanol (450 ml) is added diethyl malonate (11.7 ml, 77.2 mmol) and piperidine (7.6 ml, 77.2 mmol). The reaction mixture is stirred at RT overnight. The reaction mixture is then cooled to 0° C. using a ice/water bath and the formed precipitate is filtered and washed with ethanol. The mother liquor are concentrated and purified using flash chromatography (eluent ethyl acetate/Hexanes 3/7) to yield 7-methoxy-2-oxo-8-propyl-2H-chromene-3-carboxylic acid ethyl ester.

### e) Preparation of 7-methoxy-2-oxo-8-propyl-2H-chromene-3-carboxylic acid

To a solution of 7-methoxy-2-oxo-8-propyl-2H-chromene-3-carboxylic acid ethyl ester (15.4 g, 53.0 mmol) in THF (300 ml) is added at 0° C a 1N solution of NaOH (120 ml), the reaction mixture is then stirred overnight at RT. The reaction mixture is cooled to 0° C. using an ice/water bath and the pH was brought down to 1 using a 1N HCl solution. The reaction mixture is stirred at 0° C. for 30 minutes and the formed precipitate is filtered and washed with water. 7-methoxy-2-oxo-8-propyl-2H-chromene-3-carboxylic is isolated after drying the precipitate.

### f) Preparation of {4-[(7-methoxy-2-oxo-8-propyl-2H-chromene-3-carbonyl)-amino]-benzyl}-carbamic acid tert-butyl ester

To a solution of 7-methoxy-2-oxo-8-propyl-2H-chromene-3-carboxylic acid (600 mg, 2.28 mmol) in CH₂Cl₂ (20 ml) is added diisopropyl ethyl amine (530 ul, 3 mmol), and a 50% solution of propane phosphonic anhydride in ethyl acetate (2.9 ml, 4.57 mmol), the reaction mixture is then stirred for 30 minutes at RT. (4-Amino-benzyl)-carbamic acid tert-butyl ester (1.0 mg, 4.57 mmol) is added to the reaction mixture which is stirred at room temperature for 1 hour. The reaction mixture is then poured into an ice/water (50 ml) mixture and CH₂Cl₂ (50 ml) is added, the organic layer is separated and washed with 50 ml of brine and 50 ml of water, dried and concentrated. {4-[(7-Methoxy-2-oxo-8-propyl-2H-chromene-3-carbonyl)-amino]-benzyl}-carbamic acid tert-butyl ester is isolated by filtration of the precipitate obtained by addition of hexanes.

### g) Preparation of 7-methoxy-2-oxo-8-propyl-2H-chromene-3-carboxylic acid (4-aminomethyl-phenyl)-amide

To a solution of {4-[(7-methoxy-2-oxo-8-propyl-2H-chromene-3-carbonyl)-amino]-benzyl}-carbamic acid tert-butyl ester (640 mg, 1.37 mmol) in CH₂Cl₂/MeOH (40 ml) is added a 4M solution of HCl in dioxane. The reaction mixture is stirred at RT for 5 hours. CH₂Cl₂ (500 ml) and water (200 ml) are then added, the organic layer is separated and washed with 100 ml of a saturated solution of sodium carbonate and 100 ml of brine. 7-Methoxy-2-oxo-8-propyl-2H-chromene-3-carboxylic acid (4-aminomethyl-phenyl)-amide is isolated after precipitation using hexanes (M+H−NH₃)⁺=350.

### EXAMPLE 2

### 7-(2-Fluoro-ethoxy)-2-oxo-8-propyl-2H-chromene-3-carboxylic acid (4-methylaminomethyl-phenyl)-amide

| R₁ | R₂ | R₃ | R₄ | R₅ | (M + H)⁺ |
|---|---|---|---|---|---|
| propyl | —OCH2CH2F | H | 4-CH2NHCH3 | H | 413 |

### a) 2-Propyl-benzene-1,3-diol is prepared as described in Synthetic Communications, 20(12), 1869-1876.

### b) Preparation of 2,4-dihydroxy-3-propyl-benzaldehyde

To a solution of 2-propyl-benzene-1,3-diol (13 g, 85.4 mmol) in DMF (70 ml) is added dropwise at 0° C. under inert atmosphere a solution of phosphorous oxychloride (17.4 ml, 188 mmol) in DMF (70 ml). The reaction mixture is then stirred at RT for 1 hour. The reaction mixture is then cooled to 0° C. and quenched carefully with water. The reaction mixture is partitioned between 200 ml of ethyl acetate and 150 ml of water and the layers are separated. The organic layer is washed with 150 ml of brine, dried and concentrated. 2,4-dihydroxy-3-propyl-benzaldehyde is isolated after purification using flash chromatography (eluent Hexanes/Ethyl acetate 9/1).

### c) Preparation of 4-(2-fluoro-ethoxy)-2-hydroxy-3-propyl-benzaldehyde

To a solution of 2,4-dihydroxy-3-propyl-benzaldehyde (4 g, 22.2 mmol) in THF (50 ml) is added 1-bromo-2-fluoroethane (5.75 g, 44.4 mmol) and potassium carbonate (4.60 g, 33.3 mmol). The reaction mixture is then stirred at 80° C. for 12 hours. The reaction mixture is then concentrated and partitioned between 200 ml of ethyl acetate and 150 ml of water and the layers are separated. The organic layer is washed with 150 ml of brine and 150 ml of water, dried and concentrated. 4-(2-Fluoro-ethoxy)-2-hydroxy-3-propyl-benzaldehyde is isolated after purification using flash chromatography (eluent Hexanes/Ethyl acetate 9/1).

### d) Preparation of 7-(2-fluoro-ethoxy)-2-oxo-8-propyl-2H-chromene-3-carboxylic acid ethyl ester

To a solution of 4-(2-fluoro-ethoxy)-2-hydroxy-3-propyl-benzaldehyde (2.41 g, 10.7 mmol) in ethanol (40 ml) is added diethyl malonate (1.62 ml, 10.7 mmol) and piperidine (1.05 ml, 10.7 mmol). The reaction mixture is then stirred at RT overnight. The reaction mixture is then concentrated and partitioned between 200 ml of ethyl acetate and 150 ml of water and the layers are separated. The organic layer is washed with 150 ml of brine and 150 ml of water, dried and concentrated. 7-(2-Fluoro-ethoxy)-2-oxo-8-propyl-2H-chromene-3-carboxylic acid ethyl ester is isolated after purification using flash chromatography (eluent Hexanes/Ethyl acetate 9/1).

### e) Preparation of 7-(2-fluoro-ethoxy)-2-oxo-8-propyl-2H-chromene-3-carboxylic acid

To a solution of 7-(2-fluoro-ethoxy)-2-oxo-8-propyl-2H-chromene-3-carboxylic acid ethyl ester (2.9 g, 9.11 mmol) in THF (30 ml) is LiOH (772 mg, 18.2 mmol). The reaction mixture is then stirred at RT overnight. The reaction mixture is then concentrated and partitioned between 100 ml of ethyl acetate and 80 ml of 2N HCl and the layers are separated. The organic layer is washed with 150 ml of brine and 150 ml of water, dried and concentrated. 7-(2-Fluoro-ethoxy)-2-oxo-8-propyl-2H-chromene-3-carboxylic acid is isolated after precipitation from Hexanes/Ethyl acetate.

### f) Preparation of methyl-(4-nitro-benzyl)-amine

To a solution of 1-bromomethyl-4-nitro-benzene (6 g, 27.8 mmol) in THF (50 ml) is added triethyl amine (5 ml, 36.14 mmol) and a solution of 2N methyl amine in THF (42 ml, 84.0 mmol). The reaction mixture is then stirred at RT overnight. The reaction mixture is then concentrated and partitioned between 200 ml of ethyl acetate and 150 ml of water and the layers are separated. The organic layer is washed with 150 ml of brine and 150 ml of water, dried and concentrated. Methyl-(4-nitro-benzyl)-amine is isolated after precipitation from Hexanes/Ethyl acetate.

### g) Preparation of methyl-(4-nitro-benzyl)-carbamic acid tert-butyl ester

To a solution of methyl-(4-nitro-benzyl)-amine (6 g, 32.5 mmol) in CH₂Cl₂ (80 ml) is added diisopropyl ethyl amine (9.27 ml, 54.1 mmol) and Boc anhydride (15.8 g, 72.4 mmol). The reaction mixture is then stirred at RT overnight. The reaction mixture is partitioned with and 150 ml of water and the layers are separated. The organic layer is washed with 150 ml of brine and 150 ml of water, dried and concentrated. Methyl-(4-nitro-benzyl)-carbamic acid tert-butyl ester is isolated after purification using flash chromatography (eluent Hexanes/Ethyl acetate 9/1).

### h) Preparation of methyl (4-amino-benzyl)-methyl-carbamic acid tert-butyl ester

To a solution of methyl-(4-nitro-benzyl)carbamic acid tert-butyl ester (6.68 g, 17.6 mmol) in ethanol (40 ml) is added under inert atmosphere Pd/C (10%).The reaction mixture is placed for 2 hours under 50 psi of hydrogen atmosphere. The reaction mixture is filtered over celite and concentrated. (4-Amino-benzyl)-methyl-carbamic acid tert-butyl ester is isolated after purification using flash chromatography (eluent Hexanes/Ethyl acetate 9/1).

### i) Preparation of (4-{[7-(2-fluoro-ethoxy)-2-oxo-8-propyl-2H-chromene-3-carbonyl]-amino}-benzyl)-methyl-carbamic acid tert-butyl ester

To a solution of 7-(2-fluoro-ethoxy)-2-oxo-8-propyl-2H-chromene-3-carboxylic acid (90 mg, 0.30 mmol) in CH₂Cl₂ (5 ml) is added diisopropyl ethyl amine (94 ul, 0.39 mmol) and propylphosphonic anhydride (160 ul, 0.39 mmol). The reaction mixture is stirred at RT for 30 minutes, followed by addition of methyl (4-amino-benzyl)-methyl-carbamic acid tert-butyl ester (73.8 mg, 0.30 mmol), reaction mixture is stirred overnight at RT. The reaction mixture is partitioned with and 30 ml of water and the layers are separated. The organic layer is washed with 50 ml of brine and 50 ml of water, dried and concentrated. (4-{[7-(2-Fluoro-ethoxy)-2-oxo-8-propyl-2H-chromene-3-carbonyl]-amino}-benzyl)-methyl-carbamic acid tert-butyl ester is isolated after precipitation with Hexanes/Ethyl acetate.

### j) Preparation of 7-(2-Fluoro-ethoxy)-2-oxo-8-propyl-2H-chromene-3-carboxylic acid (4-methylaminomethyl-phenyl)-amide

A solution of 4M HCl in dioxane (3 ml) is added to (4-{[7-(2-fluoro-ethoxy)-2-oxo-8-propyl-2H-chromene-3-carbonyl]-amino}-benzyl)-methyl-carbamic acid tert-butyl ester (100 mg, 0.22 mmol). The reaction mixture is stirred at RT overnight. The reaction mixture is concentrated and 7-(2-fluoro-ethoxy)-2-oxo-8-propyl-2H-chromene-3-carboxylic acid (4-methylaminomethyl-phenyl)-amide is isolated by filtration after addition of hexanes (M+H)⁺=413.

All the following examples are synthesized according to one of the two procedures described above.

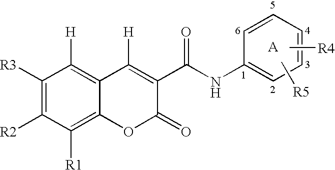

| Ex | R₁ | R₂ | R₃ | R₄ | R₅ | (M + H)⁺ |
|---|---|---|---|---|---|---|
| 3. | propyl | ethoxy | H | 4-CH2NH2 | 2-CF3 | 449.2 |
| 4. | propyl | methoxy | H | 4-CH2NH2 | 3-methyl | 364 |
| | | | | | | (M + H − NH₃)+ |
| 5. | propyl | ethoxy | H | 4-CH2NH2 | 3-CF3 | 449 |
| 6. | propyl | ethoxy | H | 4-CH2NHCH3 | H | 395 |
| 7. | propyl | methoxy | H | 4-CH2NH2 | 3-Cl | 423 (M + Na)⁺ |
| 8. | propyl | ethoxy | H | 4-CH2NH2 | 3-Cl | 437 (M + Na)+ |
| 9. | propyl | ethoxy | H | 4-CH2NH2 | H | 381 |
| 10. | allyl | methoxy | H | 4-CH2NH(CH2)2COOH | H | 437.4 |
| 11. | propyl | methoxy | H | 4-CH2NH2 | 2-methoxy | 397 |
| 12. | propyl | methoxy | H | 4-CH2NH2 | 2-methyl | 381 |
| 13. | propyl | methoxy | H | 4-CH2NH2 | 2-CF3 | 435.7 |
| 14. | propyl | methoxy | H | 4-CH2NH2 | 3-CF3 | 433 (M − H)⁻ |
| 15. | propyl | methoxy | H | 4-CH2NH2 | 2-Cl | 402.9 |
| 16. | propyl | ethoxy | H | 4-C(CH3)2NH2 | H | 410 |
| 17. | propyl | methoxy | H | 4-CH2NH2 | 2-OCF3 | 451 |
| 18. | propyl | —O-(R)-CH(CH3)CH2CH3 | H | 4-pyrrolidin-1-ylmethyl- | H | 463.2 |
| 19. | propyl | methoxy | H | 4-(1-amino-cyclopropyl)- | H | 376 |
| | | | | | | (M + H − NH₃)+ |
| 20. | propyl | ethoxy | H | 4-(1-amino-cyclopropyl)- | H | 407 |
| 21. | propyl | methoxy | H | 4-C(CH3)2NH2 | H | 396 |
| 22. | propyl | ethoxy | H | 4-CH2N(CH3)2 | H | 409.5 |
| 23. | propyl | ethoxy | H | 4-pyrrolidin-1-ylmethyl- | H | 435.1 |
| 24. | isopropoxy | methoxy | H | 4-CH2NH2 | 2-OCF3 | 467 |
| 25. | propyl | ethoxy | H | 4-CH2NH2 | 2-OCF3 | 448.1 |
| | | | | | | (M + H − NH₃)+ |
| 26. | propyl | methoxy | H | 4-CH2NHCH2CH3 | H | 395.5 |
| 27. | propyl | methoxy | H | 4-CH2NHCH3 | H | 381 |
| 28. | propyl | methoxy | H | 4-CH2NH(CH2)2OH | H | 411 |
| 29. | propyl | methoxy | H | 4-CH2NHcyclopropyl | H | 407.5 |
| 30. | propyl | methoxy | H | 4-C(CH3)2NHCH3 | H | 378 |
| | | | | | | (M + H − |
| | | | | | | NH₂CH₃)⁺ |
| 31. | allyl | methoxy | H | 4-CH2NH(CH2)2COOCH3 | H | 451.4 |
| 32. | propoxy | methoxy | H | 4-CH2NH2 | H | 383 |
| 33. | propyl | methoxy | H | 4-CH2N(CH3)(CH2)2OH | H | 425 |
| 34. | propyl | methoxy | H | 4-CH2NHcyclobutyl | H | 421.5 |
| 35. | propyl | methoxy | H | 4-pyrrolidin-1-ylmethyl- | H | 421.4 |
| 36. | allyl | methoxy | H | 4-CH2N(CH3)2 | H | 393.4 |
| 37. | propyl | methoxy | H | 4-CH2N(CH3)2 | H | 395 |
| 38. | propyl | methyl | H | 4-CH2NH2 | H | 351.2 |
| 39. | propyl | methoxy | H | 4-(1-methylamino-cyclopropyl)- | H | 407 |
| 40. | propyl | methoxy | H | 4-morpholin-4-ylmethyl- | 2-OCF3 | 521 |
| 41. | allyl | methoxy | H | 4-piperidin-1-ylmethyl- | H | 433.4 |
| 42. | propyl | methoxy | H | 4-CH2NHCH2phenyl | H | 457.6 |
| 43. | isopropoxy | methoxy | H | 4-CH2NH2 | H | 383 |
| 44. | propyl | methoxy | H | 4-piperidin-1-ylmethyl- | H | 435.5 |
| 45. | ethoxy | methoxy | H | 4-CH2NH2 | H | 369 |
| 46. | propyl | methoxy | H | 4-CH₂CH₂NH₂ | H | 381.4 |
| 47. | propyl | methoxy | H | 4-CH2NH2 | 3-methoxy | 380 |
| | | | | | | (M + H − NH₃)+ |
| 48. | allyl | methoxy | H | 4-CH2NH(CH2)2COOC(CH3)3 | H | 193.5 |
| 49. | propyl | methoxy | H | 4-C(CH3)2N(CH3)2 | H | 423 |
| 50. | propyl | methoxy | Cl | 4-CH2NH2 | H | 423 (M + Na)+ |
| 51. | isopropoxy | isopropoxy | H | 4-CH2NH2 | H | 394 |
| | | | | | | (M + H − NH₃)+ |
| 52. | propyl | methoxy | H | 4-CH2NH2 | 2-OH | 366 |
| | | | | | | (M + H − NH₃)+ |
| 53. | t-butyl | H | t-butyl | 4-CH2NH2 | H | 407 |
| 54. | propyl | methoxy | H | 4-CH₂-(S)-CH(NH₂)COOH | H | 425 |
| 55. | propyl | methoxy | H | 4-CH2NHCH(CH3)2 | H | 409.2 |
| 56. | propyl | methoxy | H | 4-CH2NH(CH2)2OCH3 | H | 425 |
| 57. | allyl | methoxy | H | 4-CONH(CH2)2COOH | H | 451 |
| 58. | t-butyl | H | t-butyl | 4-pyrrolidin-1-ylmethyl- | H | 461 |
| 59. | propyl | methoxy | H | 4-CH₂-(R)-CH(CH3)NH₂ | H | 395.4 |
| 60. | propyl | methoxy | H | 4-CH2NH2 | 3-OH | 366 |
| | | | | | | (M + H − NH₃)⁺ |
| 61. | allyl | methoxy | H | 4-CH2NHacetyl | H | 407 |
| 62. | t-butyl | H | t-butyl | 4-CH2NH2 | 2-OCF3 | 491 |
| 63. | propyl | methoxy | H | 4-CH2N(CH3)2 | 2-OCF3 | 479 |
| 64. | propyl | ethoxy | H | 4-morpholin-4-ylmethyl- | H | 451 |
| 65. | propyl | methoxy | H | 4-CH2N(CH3)CH2CCH | H | 419 |
| 66. | allyl | H | H | 4-CH2NH(CH2)2COOCH3 | H | 407 |
| 67. | methoxy | methoxy | H | 4-CH2NH2 | H | 338 |
| | | | | | | (M + H − NH₃)+ |
| 68. | propyl | methoxy | H | 4-morpholin-4-ylmethyl- | H | 437 |
| 69. | propyl | methoxy | H | 4-CH2NHCH2CON(CH3)2 | H | 452 |
| 70. | propyl | methoxy | H | 4-CH2NHC(CH3)3 | H | 423.4 |
| 71. | allyl | methoxy | H | 4-morpholin-4-ylmethyl- | H | 435 |
| 72. | propyl | methoxy | H | 4-CH₂N((CH2)₂OCH3)₂ | H | 483.1 |
| 73. | propyl | methoxy | H | 3-CH2NHCH3 | H | 381 |
| 74. | propyl | methoxy | H | 4-((2R,6S)-2,6-dimethyl- | H | 465 |
| | | | | morpholin-4-ylmethyl)- | | |
| 75. | methyl | H | —OCF3 | 4-CH2N(CH3)2 | H | 421 |
| 76. | propyl | trifluoromethyl | H | 4-morpholin-4-ylmethyl- | H | 475.5 |
| 77. | propyl | methoxy | H | 4-C(CH3)₂morpholine | H | 465 (M − H)− |
| 78. | allyl | methoxy | H | 4-(2-oxo-pyrrolidin-1-ylmethyl)- | H | 433 |
| 79. | propyl | methoxy | H | 4-(1-morpholin-4-yl-ethyl)- | H | 451.5 |
| 80. | propyl | methoxy | H | 3-(CH₂)₂NH₂ | H | 381 |
| 81. | H | ethoxy | allyl | 4-CH2N(CH3)2 | H | 393 |
| 82. | propyl | methoxy | H | 4-(1-dimethylamino-cyclopropyl)- | H | 421 |
| 83. | propyl | methoxy | H | 4-(4-methyl-piperazin-1-ylmethyl)- | H | 450.4 |
| 84. | propyl | methoxy | H | 4-CH2NH(CH2)2CF3 | H | 463 |
| 85. | H | methoxy | H | 4-CH2NHCH3 | H | 339 |
| 86. | allyl | H | H | 4-CH2N(CH3)2 | H | 363.5 |
| 87. | propyl | methoxy | H | 4-CNN(CH3)₂ | H | 408.5 |
| 88. | propyl | methoxy | H | 3-piperazin-1-ylmethyl- | H | 436 |
| 89. | propyl | methoxy | H | 4-C(CH3)NOH | H | 395 |
| 90. | allyl | methoxy | H | 4-CH2NHCOO-t-butyl | H | 465 |
| 91. | H | methoxy | H | 4-CH2NH2 | H | 325 |
| 92. | propyl | methoxy | H | 4-CH2NHCH3 | 2-OCF3 | 465 |
| 93. | propyl | methoxy | H | 4-(S,S)- | H | 455 |
| | | | | CH(OH)CH(CH2OH)N(CH3)2 | | |
| 94. | propyl | methoxy | H | 4-(R,R)-CH(OH)CH(CH₂OH)NH₂ | H | 427 |
| 95. | propyl | methoxy | H | 4-(S,S)-CH(OH)CH(CH₂OH)NH₂ | H | 427 |
| 96. | propyl | methoxy | H | 4-(4-oxy-morpholin-4-ylmethyl)- | H | 453 |
| 97. | propyl | —O-(R)-CH(CH3)CH2CH3 | H | 4-CH2NH2 | H | 409.2 |
| 98. | propyl | —O-(S)-CH(CH3)CH2CH3 | H | 4-CH2NH2 | H | 409 |
| 99. | propyl | —OCH2CH2F | H | 4-CH2NH2 | H | 399 |
| 100. | propyl | —OCH2CHF2 | H | 4-CH2NH2 | H | 400 |
| | | | | | | (M + H − NH3)+ |
| 101. | propyl | isopropoxy | H | 4-CH2NH2 | H | 378.3 |
| 102. | propyl | —OCH2CHF2 | H | 4-C(CH3)2NH2 | H | 446 |
| 103. | propyl | —O-(R)-CH(CH3)CH2CH3 | H | 4-CH2NHCH3 | H | 423 |
| 104. | propyl | —O-(S)-CH(CH3)CH2CH3 | H | 4-CH2NHCH3 | H | 423 |
| 105. | propyl | butoxy | H | 4-CH2NH2 | H | 395 |
| 106. | propyl | isopropoxy | H | 4-CH2NH2 | 2-OCF3 | 479 |
| 107. | propyl | isopropoxy | H | 4-CH2NHCH3 | H | 409 |
| 108. | propyl | —OCH2CH(CH3)2 | H | 4-CH2NH2 | H | 409 |
| 109. | propyl | —O-(R)-CH(CH3)CH2CH3 | H | 4-CH2N(CH3)2 | H | 437 |
| 110. | propyl | isopropoxy | H | 4-C(CH3)2NH2 | H | 406 |
| | | | | | | (M + H − NH3)+ |
| 111. | propyl | —O-(S)-CH(CH3)CH2CH3 | H | 4-pyrrolidin-1-ylmethyl- | H | 463 |
| 112. | propyl | —O-(S)-CH(CH3)CH2CH3 | H | 4-C(CH3)2NH2 | H | 438.2 |
| 113. | propyl | —OCH2cyclopropyl | H | 4-CH2NH2 | H | 407 |
| 114. | propyl | —Ocyclobutyl | H | 4-CH2N(CH3)2 | H | 435 |
| 115. | propyl | —OCH2CHF2 | H | 4-CH2NHCH3 | H | 431 |
| 116. | propyl | —Ocyclopentyl | H | 4-CH2NH2 | H | 421 |
| 117. | propyl | —OCH(CH3)cyclopropyl | H | 4-CH2N(CH3)2 | H | 449 |
| 118. | propyl | —O-(S)-CH(CH3)CH2CH3 | H | 4-CH2N(CH3)2 | H | 437 |
| 119. | propyl | —OCH2CH2F | H | 4-pyrrolidin-1-ylmethyl- | H | 453.2 |
| 120. | propyl | isopropoxy | H | 4-CH2N(CH3)2 | H | 423.2 |
| 121. | propyl | —OCH2cyclopropyl | H | 4-CH2N(CH3)2 | H | 435 |
| 122. | propyl | —OCH(CH3)CH(CH3)2 | H | 4-CH2N(CH3)2 | H | 451.2 |
| 123. | propyl | —OCH2CH(CH3)2 | H | 4-CH2N(CH3)2 | H | 437 |
| 124. | propyl | —O-(S)-CH(CH3)CH2CH3 | H | 4-morpholin-4-ylmethyl- | H | 479.24 |
| 125. | propyl | —Ocyclopentyl | H | 4-CH2N(CH3)2 | H | 449.72 |
| 126. | propyl | isopropoxy | H | 4-morpholin-4-ylmethyl- | H | 465.4 |
| 127. | propyl | —OCH2CH(CH3)2 | H | 4-morpholin-4-ylmethyl- | H | 479 |
| 128. | propyl | —Ocyclopentyl | H | 4-morpholin-4-ylmethyl- | H | 491 (M − H)− |
| 129. | propyl | methoxy | H | 4-CH2N(CH3)(CH2)2OCH3 | H | 439 |
| 130. | propyl | methoxy | H | 4-morpholin-4-ylmethyl- | 2-CF3 | 505 |
| 131. | propyl | methoxy | H | 4-morpholin-4-ylmethyl- | 2-Cl | 471 |
| 132. | propyl | ethoxy | H | 4-CH2N(CH3)(CH2)2OCH3 | H | 453.2 |
| 133. | allyl | ethyl | H | 4-CH2N(CH3)2 | H | 391.2 |
| 134. | ethyl | methoxy | H | 4-CH2NH2 | H | 336 [MH − NH3]+ |
| 135. | propyl | methoxy | H | 4-morpholin-4-ylmethyl- | 2-methoxy | 380 |
| | | | | | | [MH-morpholine]+ |
| 136. | propyl | methoxy | H | 4-morpholin-4-ylmethyl- | 3-Cl | 471 |
| 137. | isopropyl | methoxy | H | 4-CH2N(CH3)2 | H | 395.2 |
| 138. | propyl | methoxy | H | 4-CH2-(R)-CH(NH2)COOH | H | 422.9 [M − H] |
| 139. | propyl | methoxy | H | 4-CH2-(R)-CH(NH2)CONH2 | H | 424 |
| 140. | propyl | methoxy | H | 4-CH2-(R)-CH(NH2)CON(CH3)2 | H | 452 |
| 141. | allyl | isopropyl | H | 4-CH2N(CH3)2 | H | 405.6 |
| 142. | propyl | ethyl | H | 4-CH2N(CH3)2 | H | 393.3 |
| 143. | butyl | methoxy | H | 4-CH2NH2 | H | 364 [MH − NH3]+ |
| 144. | propyl | ethoxy | H | 4-(3,3-difluoro-piperidin-1- | H | 485.2 |
| | | | | ylmethyl)- | | |
| 145. | propyl | methoxy | H | 4-(3,3-difluoro-pyrrolidin-1- | H | 457.2 |
| | | | | ylmethyl)- | | |
| 146. | propyl | ethoxy | H | 4-(3,3-difluoro-pyrrolidin-1- | H | 471.2 |
| | | | | ylmethyl)- | | |
| 147. | propyl | ethyl | H | 4-CH2NH2 | H | 365.2 |
| 148. | —(CH2)2CH(CH3)2 | methoxy | H | 4-CH2N(CH3)2 | H | 423.3 |
| 149. | allyl | methoxy | H | 4-CH2NH2 | H | 365 |
| 150. | —(CH2)2CH(CH3)2 | methoxy | H | 4-morpholin-4-ylmethyl- | H | 465.3 |
| 151. | —CH2CH(CH3)2 | methoxy | H | 4-CH2N(CH3)2 | H | 409.3 |
| 152. | —CH2CH(CH3)2 | methoxy | H | 4-morpholin-4-ylmethyl- | H | 451.3 |
| 153. | propyl | ethyl | H | 4-C(CH3)2NH2 | H | 393.3 |
| 154. | —CH2CH2Ph | methoxy | H | 4-CH2NH2 | H | 412 [MH − NH3]+ |
| 155. | propyl | isopropyl | H | 4-CH2NH2 | H | 379.2 |
| 156. | ethyl | methoxy | H | 4-C(CH3)2NH2 | H | 364 [MH − NH3]+ |
| 157. | propyl | methoxy | H | 4-CH2C(CH3)2NH2 | H | 409.3 |
| 158. | propyl | isopropyl | H | 4-C(CH3)2NH2 | H | 407.9 |
| 159. | ethyl | methoxy | H | 4-(1-amino-cyclopropyl)- | H | 362 [MH − NH3]+ |
| 160. | —CH2CH2OH | methoxy | H | 4-CH2NH2 | H | 352 [MH − NH3]+ |
| 161. | propyl | methoxy | H | 4-CH2N(CH3)CH2CH3 | H | 409.3 |
| 162. | propyl | ethoxy | H | 4-CH2N(CH3)CH2CH3 | H | 423.3 |
| 163. | —(CH2)2CH(CH3)2 | methoxy | H | 4-CH2NH2 | H | 378.3 |
| | | | | | | [(MH − NH3)]+ |
| 164. | —CH2CH(CH3)2 | methoxy | H | 4-CH2NH2 | H | 364.3 |
| | | | | | | [(MH − NH3)]+ |
| 165. | —(CH2)2CH(CH3)2 | methoxy | H | 4-C(CH3)2NH2 | H | 406.3 |
| | | | | | | [(MH − NH3)]+ |
| 166. | —CH2CH(CH3)2 | methoxy | H | 4-C(CH3)2NH2 | H | 392.3 |
| | | | | | | [(MH − NH3)]+ |
| 167. | propyl | methoxy | H | 4-C(CH3)2CH2NH2 | H | 409 |
| 168. | butyl | methoxy | H | 4-(1-amino-cyclopropyl)- | H | 390 [MH − NH3]+ |
| 169. | propyl | methoxy | H | 4-(1-aminomethyl-cyclopropyl)- | H | 407 |
| 170. | butyl | methoxy | H | 4-C(CH3)2NH2 | H | 392 [MH − NH3]+ |
| 171. | butyl | methoxy | H | 4-CH2N(CH3)2 | H | 364 [MH − NMe2]+ |
| 172. | butyl | methoxy | H | 4-pyrrolidin-1-ylmethyl- | H | 364 |
| | | | | | | [MH-cy- |
| | | | | | | pentylamin]+ |
| 173. | propyl | methoxy | H | 4-((S)-3-hydroxy-pyrrolidin-1- | H | 437.3 |
| | | | | ylmethyl)- | | |
| 174. | propyl | ethoxy | H | 4-((S)-3-hydroxy-pyrrolidin-1- | H | 451.3 |
| | | | | ylmethyl)- | | |
| 175. | propyl | methoxy | H | 4-((R)-3-hydroxy-pyrrolidin-1- | H | 437.3 |
| | | | | ylmethyl)- | | |
| 176. | propyl | ethoxy | H | 4-((R)-3-hydroxy-pyrrolidin-1- | H | 451.2 |
| | | | | ylmethyl)- | | |
| 177. | —CH2CH(CH3)2 | methoxy | H | 4-(1-amino-cyclopropyl)- | H | 390.3 |
| | | | | | | [(MH − NH3)]+ |
| 178. | butyl | methoxy | H | 4-morpholin-4-ylmethyl- | H | 364 |
| | | | | | | [MH − |
| | | | | | | morpholine]+ |
| 179. | ethyl | ethoxy | H | 4-(1-amino-cyclopropyl)- | H | 376.3 |
| | | | | | | [(MH − NH3)]+ |
| 180. | ethyl | ethoxy | H | 4-C(CH3)2NH2 | H | 378.3 |
| | | | | | | [(MH − NH3)]+ |
| 181. | butyl | methoxy | H | 4-CH2NHCH3 | H | 364 |
| | | | | | | [MH − |
| | | | | | | (NH2—CH3)]+ |
| 182. | ethyl | methoxy | H | 4-CH2N(CH3)2 | H | 336 [MH − NMe2]+ |
| 183. | ethyl | methoxy | H | 4-CH2NHCH3 | H | 336 |
| | | | | | | [MH − |
| | | | | | | (NH2—CH3)]+ |
| 184. | —CH2CH2CH2CH2— | | H | 4-CH2NH2 | H | 332 |
| | | | | | | [HMH − NH3]+ |
| 185. | propyl | methoxy | H | 4-CH2N(CH3)cyclopropyl | H | 421.3 |
| 186. | propyl | ethoxy | H | 4-CH2N(CH3)cyclopropyl | H | 435.3 |
| 187. | propyl | methyl | H | 4-C(CH3)2NH2 | H | 362.2 |
| | | | | | | [MH − NH3]+ |
| 188. | propyl | ethoxy | H | 4-CH2NHcyclopropyl | H | 421.3 |
| 189. | propyl | methyl | H | 4-(1-amino-cyclopropyl)- | H | 360.2 |
| | | | | | | [MH − NH3]+ |
| 190. | propyl | methoxy | H | 4-CH(OH)CH2NH2 | H | 379.2 |
| | | | | | | [MH − H2O]+ |
| 191. | —CH2CH2CH2CH2— | | H | 4-C(CH3)2NH2 | H | 339 [M + Na]+ |
| 192. | methyl | methoxy | H | 4-C(CH3)2NH2 | H | 350 [MH − NH3]+ |
| 193. | methyl | methoxy | H | 4-(1-amino-cyclopropyl)- | H | 348 [MH − NH3]+ |
| 194. | propyl | methoxy | H | 4-CH2-(S)-CH(NH2)CH2OH | H | 411 |
| 195. | propyl | trifluoromethyl | H | 4-(1-amino-cyclopropyl)- | H | 431.4 |
| 196. | propyl | methoxy | H | 4-CH2-(R)-CH(NH2)CH2OH | H | 411 |
| 197. | isopropoxy | methoxy | H | 4-(1-amino-cyclopropyl)- | H | 392 [MH − NH3]+ |
| 198. | isopropoxy | methoxy | H | 4-C(CH3)2NH2 | H | 394 [MH − NH3]+ |
| 199. | methyl | methoxy | H | 4-CH2NH2 | H | 322 [MH − NH3]+ |
| 200. | ethyl | methoxy | H | 4-CH2NH2 | 2-OCF3 | 420 [MH − NH3]+ |
| 201. | ethyl | methoxy | H | 4-pyrrolidin-1-ylmethyl- | H | 407 |
| 202. | ethyl | methoxy | H | 4-morpholin-4-ylmethyl- | H | 423 |
| 203. | ethyl | methoxy | H | 4-CH2NHCH(CH3)2 | H | 395.4 |
| 204. | ethyl | methoxy | H | 4-CH2N(CH3)CH2CH3 | H | 395.4 |
| 205. | ethyl | methoxy | H | 4-CH2NHCH2CH3 | H | 336.3 |
| | | | | | | [MH − EtNH2]+ |
| 206. | isopropyl | methoxy | H | 4-C(CH3)2NH2 | H | 378.3 |
| | | | | | | [(MH − NH3)]+ |
| 207. | isopropyl | methoxy | H | 4-(1-amino-cyclopropyl)- | H | 376.3 |
| | | | | | | [(MH − NH3)]+ |
| 208. | ethyl | methoxy | H | 4-(S)-CH(CH3)NH2 | H | 350 [MH − NH3]+ |
| 209. | ethyl | methoxy | H | 4-(R)-CH(CH3)NH2 | H | 350 [MH − NH3]+ |
| 210. | ethyl | methoxy | H | 4-(S,S)- | H | 441 |
| | | | | CH(OH)CH(CH2OH)N(CH3)2 | | |
| 211. | ethyl | methoxy | H | 4-(S,S)-CH(OH)CH(CH₂OH)NH₂ | H | 413 |
| 212. | ethyl | methoxy | H | 4-CH2NH2 | 2-methoxy | 383 |
| 213. | ethyl | methoxy | H | 4-((S)-3-hydroxy-pyrrolidin-1- | H | 423.3 |
| | | | | ylmethyl)- | | |
| 214. | ethyl | methoxy | H | 4-((R)-3-hydroxy-pyrrolidin-1- | H | 423.3 |
| | | | | ylmethyl)- | | |
| 215. | ethyl | methoxy | H | 4-CH2NHcyclopropyl | H | 393.3 |
| 216. | ethyl | methoxy | H | 4-CH2NH(CH2)2OH | H | 397.3 |
| 217. | isopropyl | methoxy | H | 4-CH2NHCH3 | H | 350.3 |
| | | | | | | [MH − NH2CH3]+ |
| 218. | isopropyl | methoxy | H | 4-pyrrolidin-1-ylmethyl- | H | 421.3 |
| 219. | isopropyl | methoxy | H | 4-CH2N(CH3)CH2CH3 | H | 409.4 |
| 220. | ethyl | methoxy | H | 4-CH2N(CH3)(CH2)2OH | H | 411.3 |
| 221. | ethyl | methoxy | H | 4-CH2-(R)-CH(NH2)CH2OH | H | 379.1 |
| 222. | ethyl | methoxy | H | 4-(1-acetylamino-cyclopropyl)- | H | 421 |
| 223. | propyl | methoxy | H | 4-(1-acetylamino-cyclopropyl)- | H | 435.1 |
| 224. | propyl | —OCH2CHF2 | H | 4-CH2N(CH3)2 | H | 445 |
| 225. | propyl | —OCH2CHF2 | H | 4-CH2N(CH3)(CH2)2OCH3 | H | 489.2 |
| 226. | propyl | —OCH2CH2F | H | 4-CH2N(CH3)(CH2)2OMe | H | 471 |
| 227. | propyl | —OCH2CHF2 | H | 4-morpholin-4-ylmethyl- | H | 487.2 |
| 228. | propyl | —OCH2CH2F | H | 4-CH2N(CH3)2 | H | 427.1 |
| 229. | propyl | —OCH2CH2F | H | 4-morpholin-4-ylmethyl- | H | 469.2 |
| 230. | propyl | —OCH2CHF2 | H | 4-(1-amino-cyclopropyl)- | H | 426.1 |
| | | | | | | [MH − NH3]+ |
| 231. | propyl | —OCH2CH2F | H | 4-(1-amino-cyclopropyl)- | H | 425.3 |
| 232. | propyl | —O-(S)-CH(CH3)CH2CH3 | H | 4-(1-amino-cyclopropyl)- | H | 418.1 |
| | | | | | | [MH − NH3]+ |
| 233. | propyl | —OCH2CH2F | H | 4-C(CH3)2NH2 | H | 428.3 |
| 234. | propyl | —OCH2CH2F | H | 4-(3,3-difluoro-piperidin-1- | H | 503.2 |
| | | | | ylmethyl)- | | |
| 235. | propyl | —OCH2CHF2 | H | 4-(3,3-difluoro-piperidin-1- | H | 521.2 |
| | | | | ylmethyl)- | | |
| 236. | propyl | —O-(S)-CH(CH3)CH2CH3 | H | 4-(3,3-difluoro-piperidin-1- | H | 513.3 |
| | | | | ylmethyl)- | | |
| 237. | propyl | —OCH2CH2F | H | 4-(3,3-difluoro-pyrrolidin-1- | H | 489.2 |
| | | | | ylmethyl)- | | |
| 238. | propyl | —OCH2CHF2 | H | 4-(3,3-difluoro-pyrrolidin-1- | H | 507.2 |
| | | | | ylmethyl)- | | |
| 239. | propyl | —O-(S)-CH(CH3)CH2CH3 | H | 4-(3,3-difluoro-pyrrolidin-1- | H | 499.3 |
| | | | | ylmethyl)- | | |
| 240. | propyl | —OCH2CH2F | H | 4-CH2N(CH3)CH2CH3 | H | 441.3 |
| 241. | propyl | —OCH2CHF2 | H | 4-CH2N(CH3)CH2CH3 | H | 459.3 |
| 242. | propyl | —OCH2CH2CF3 | H | 4-CH2NH2 | H | 432.2 [M − NH3]+ |
| 243. | propyl | —OCH2CH2CF3 | H | 4-(1-amino-cyclopropyl)- | H | 458.2 [M − NH3]+ |
| 244. | propyl | —OCH2CH2CF3 | H | 4-CH2N(CH3)2 | H | 477.3 |
| 245. | propyl | —OCH2CH2F | H | 4-CH2N(CH3)cyclopropyl | H | 453.3 |
| 246. | propyl | —OCH2CH2F | H | 4-CH2NHcyclopropyl | H | 439.2 |
| 247. | propyl | —OCH2CH2CF3 | H | 4-C(CH3)2NH2 | H | 460.3 |
| | | | | | | [MH − NH3]+ |
| 248. | propyl | methoxy | H | 3-CH2NH2 | H | 367 |
| 249. | propyl | methoxy | H | 4-CH2NH2 | H | 368 |
| 250. | propyl | methoxy | H | 4-CH2NH2 | H | 368 |
| 251. | methoxy | methoxy | H | 4-CH2NHCH3 | H | 369 |
| 252. | ethoxy | methyl | H | 4-(1-amino-cyclopropyl)- | H | 379.2 |
| 253. | ethyl | methoxy | H | 4-(R)-CH(CH3)NHCH3 | H | 381 |
| 254. | methoxy | methoxy | H | 4-(1-amino-cyclopropyl)- | H | 381 |
| 255. | ethyl | methoxy | H | 4-(S)-CH(CH3)NHCH3 | H | 381 |
| 256. | ethoxy | methyl | H | 4-C(CH3)2NH2 | H | 382.2 |
| 257. | ethyl | methoxy | H | 4-CH(OH)CH2NH2 enantiomer A | H | 383 |
| 258. | ethyl | methoxy | H | 4-CH(OH)CH2NH2 enantiomer B | H | 383 |
| 259. | allyl | methoxy | H | 4-(1-amino-cyclopropyl)- | H | 391 |
| 260. | ethyl | methoxy | H | 4-(1-methylamino-cyclopropyl)- | H | 393 |
| 261. | acetyl | methoxy | H | 4-(1-amino-cyclopropyl)- | H | 393.3 |
| 262. | ethoxy | methoxy | H | 4-(1-amino-cyclopropyl)- | H | 395 |
| 263. | propyl | methoxy | H | 3-CH2N(CH3)2 | H | 395 |
| 264. | nitro | methoxy | H | 4-(1-amino-cyclopropyl)- | H | 396 |
| 265. | isopropoxy | methoxy | H | 4-CH2NHCH3 | H | 397 |
| 266. | ethoxy | methoxy | H | 4-CH(OH)CH2NH2 enantiomer A | H | 399 |
| 267. | ethoxy | methoxy | H | 4-CH(OH)CH2NH2 enantiomer B | H | 399 |
| 268. | methoxy | methoxy | H | 4-CH2NH(CH2)2OH | H | 399.1 |
| 269. | ethoxy | methoxy | H | 4-(1-methylamino-cyclopropyl)- | H | 409 |
| 270. | ethyl | methoxy | H | 4-CH2N(CH3)acetyl | H | 409 |
| 271. | ethoxy | methoxy | H | 4-CH2NHcyclopropyl | H | 409.2 |
| 272. | ethyl | methoxy | H | 4-CH2N(CH3)2 | 2-methoxy | 411 |
| 273. | isopropoxy | methoxy | H | 4-CH2N(CH3)2 | H | 411 |
| 274. | —OCH2CH2OH | methoxy | H | 4-(1-amino-cyclopropyl)- | H | 411 |
| 275. | methoxy | methoxy | H | 4-CH2N(CH3)acetyl | H | 411 |
| 276. | ethoxy | methoxy | H | 4-C(CH3)2NHCH3 | H | 411 |
| 277. | isopropyl | methoxy | H | 4-CH2-(R)-CH(NH2)CH2OH | H | 411.2 |
| 278. | ethyl | methoxy | H | 4-CH2NH(CH2)2OH | 2-methyl | 411.2 |
| 279. | ethoxy | methoxy | H | 4-CH2NH(CH2)2OH | H | 413.2 |
| 280. | —OCH2cyclopropyl | methoxy | H | 4-(1-amino-cyclopropyl)- | H | 421.2 |
| 281. | isopropoxy | methoxy | H | 4-CH2NHcyclopropyl | H | 423.2 |
| 282. | ethoxy | methoxy | H | 4-pyrrolidin-1-ylmethyl- | H | 423.3 |
| 283. | —OCH2CH(CH3)2 | methoxy | H | 4-(1-amino-cyclopropyl)- | H | 423.3 |
| 284. | —OCH2cyclopropyl | methoxy | H | 4-C(CH3)2NH2 | H | 424.2 |
| 285. | —OCH2CH2OCH3 | methoxy | H | 4-(1-amino-cyclopropyl)- | H | 425 |
| 286. | ethoxy | methoxy | H | 4-(R)-CH(CH3)NHacetyl | H | 425 |
| 287. | ethoxy | methoxy | H | 4-(S)-CH(CH3)NHacetyl | H | 425 |
| 288. | isopropoxy | methoxy | H | 4-CH2NHacetyl | H | 425 |
| 289. | ethoxy | methoxy | H | 4-C(CH3)2N(CH3)2 | H | 425 |
| 290. | —OCH2CH(CH3)2 | methoxy | H | 4-C(CH3)2NH2 | H | 426.2 |
| 291. | —O-(S)-CH(CH3)CH2CH3 | methoxy | H | 4-C(CH3)2NH2 | H | 426.2 |
| 292. | —O-(R)-CH(CH3)CH2CH3 | methoxy | H | 4-C(CH3)2NH2 | H | 426.2 |
| 293. | isopropoxy | methoxy | H | 4-CH2-(R)- | H | 427 |
| | | | | CH(NH2)CH2OH | | |
| 294. | ethyl | methoxy | H | 4-(S,S)- | H | 427 |
| | | | | CH(OCH3)CH(CH₂OH)NH₂ | | |
| 295. | isopropoxy | methoxy | H | 4-CH2NH(CH2)2OH | H | 427.1 |
| 296. | ethoxy | methoxy | H | 4-CH2NH(CH2)2OH | 2-methyl | 427.1 |
| 297. | ethoxy | methoxy | H | 4-CH2N(CH3)(CH2)2OH | H | 427.3 |
| 298. | ethoxy | methoxy | H | 4-(1-acetylamino-cyclopropyl)- | H | 437 |
| 299. | ethyl | methoxy | H | 4-(2-morpholin-4-yl-ethyl)- | H | 437 |
| 300. | propyl | methoxy | H | 3-morpholin-4-ylmethyl)- | H | 437 |
| 301. | isopropoxy | methoxy | H | 4-pyrrolidin-1-ylmethyl- | H | 437.3 |
| 302. | ethoxy | methoxy | H | 4-((S)-2-oxo-oxazolidin-4- | H | 439 |
| | | | | ylmethyl)- | | |
| 303. | isopropoxy | methoxy | H | 4-CH2N(CH3)acetyl | H | 439 |
| 304. | ethoxy | methoxy | H | 4-((R)-2-oxo-oxazolidin-4- | H | 439 |
| | | | | ylmethyl)- | | |
| 305. | ethoxy | methoxy | H | 4-((S)-3-hydroxy-pyrrolidin-1- | H | 439.1 |
| | | | | ylmethyl)- | | |
| 306. | ethyl | methoxy | H | 4-CH2N(CH2CH2OH)acetyl | H | 439.2 |
| 307. | ethoxy | methoxy | H | 4-morpholin-4-ylmethyl- | H | 439.3 |
| 308. | ethyl | methoxy | H | 4-(S,S)- | H | 441 |
| | | | | CH(OCH3)CH(CH₂OCH3)NH₂ | | |
| 309. | —O-(R)-CH(CH3)CH2CH3 | methoxy | H | 4-CH2NH(CH2)2OH | H | 441.1 |
| 310. | isopropoxy | methoxy | H | 4-CH2NH(CH2)2OH | 2-methyl | 441.1 |
| 311. | —O-(S)-CH(CH3)CH2CH3 | methoxy | H | 4-CH2NH(CH2)2OH | H | 441.2 |
| 312. | ethoxy | methoxy | H | 4-CH2N(CH3)(CH2)2OH | 2-methyl | 441.3 |
| 313. | ethyl | methoxy | H | 4-CH2N(CH3)(CH2)2OH | 2-methoxy | 441.4 |
| 314. | isopropoxy | methoxy | H | 4-CH2N(CH3)(CH2)2OH | H | 441.4 |
| 315. | butyl | methoxy | H | 4-(1-acetylamino-cyclopropyl)- | H | 449 |
| 316. | propyl | methoxy | H | 4-(2-morpholin-4-yl-ethyl)- | H | 451 |
| 317. | ethoxy | methoxy | H | 4-(2-morpholin-4-yl-ethyl)- | H | 453 |
| 318. | ethoxy | methoxy | H | 4-((S)-3-hydroxy-pyrrolidin-1- | H | 453.2 |
| | | | | ylmethyl)- | | |
| 319. | ethyl | methoxy | H | 4-(S,S)- | H | 455 |
| | | | | CH(OH)CH(CH₂OCH3)N(CH3)2 | | |
| 320. | ethoxy | methoxy | H | 4-(S,S)- | H | 457 |
| | | | | CH(OH)CH(CH2OH)N(CH3)2 | | |
| 321. | isopropoxy | methoxy | H | 4-CH2N(CH3)(CH2)2OH | 2-methoxy | 471.4 |
| 322. | ethyl | methoxy | H | 4-CH2N(CH3)(CH2)2OH | 2-methyl | 4253 |
| 323. | ethoxy | methoxy | H | 4-(S)-CH(CH3)NH2 | H | 366 [M − NH3]+ |
| 324. | ethyl | methoxy | H | 4-(1-amino-cyclopentyl)- | H | 408 |
| | | | | | | [(M − NH3) + |
| | | | | | | H2O]+ |
| 325. | H | ethoxy | H | 4-(1-amino-cyclopropyl)- | H | 365 [M + H]+ |
| 326. | allyl | H | methoxy | 4-C(CH3)2NH2 | H | 376 [MH − NH3]+ |
| 327. | ethoxy | methoxy | H | 4-(R)-CH(CH3)NH2 | H | 366 [M − NH3]+ |
| 328. | ethoxy | methoxy | H | 4-(R)-CH(CH2CH3)NH2 | H | 380.2 [M − NH3]+ |
| 329. | ethoxy | methoxy | H | 4-(S)-CH(CH2CH3)NH2 | H | 394 [M − NH3]+ |
| 330. | ethoxy | methoxy | H | 4-(R)-CH(CH2CH2CH3)NH2 | H | 394 [M − NH3]+ |
| 331. | methoxy | methoxy | H | 4-C(CH3)2NH2 | H | 765 [2M + H]+ |
| 332. | allyl | methoxy | H | 4-C(CH3)2NH2 | H | 785 [2M + H]+ |
| 333. | ethoxy | methoxy | H | 4-C(CH3)2NH2 | H | 793 [2M + H]+ |
| 334. | —OCH2CH2OCH3 | methoxy | H | 4-C(CH3)2NH2 | H | 853 [2M + H]+ |
| 335. | allyl | methoxy | H | 4-CONHCH2COOH | H | |
| 336. | t-butyl | H | H | 4-(1-amino-cyclopropyl)- | H | |
| 337. | t-butyl | H | H | 4-C(CH3)2NH2 | H | |
| 338. | ethoxy | H | H | 4-(1-amino-cyclopropyl)- | H | 365.3 |
| 339. | allyl | H | H | 4-(1-amino-cyclopropyl)- | H | 361.2 |
| 340. | allyl | H | H | 4-CH2NH(CH2)2OH | H | 379.2 |

| | | | | | |
|---|---|---|---|---|---|
| 341. | propyl | methoxy | H | —CH₂—NH—CH₂—CH₂— | 393 |
| 342. | propyl | methoxy | H | —C(═NH)—NH—CH₂— | 392 |

*Ex 249: N in 3-pos of ring A
**Ex 250: N in 2-pos of ring A

The compounds of formula I in free form or in pharmaceutically acceptable salt form, exhibit valuable pharmacological properties, e.g. as S1P1 receptor agonists, e.g. as indicated in in vitro and in vivo tests and are therefore indicated for therapy.

A. In vitro

The compounds of formula I have binding affinity to individual human S1P receptors as determined in following assays:

A.1 In vitro: GPCR Activation Assay Measuring GTP [γ-³⁵S] Binding to Membranes Prepared from CHO Cells Expressing Human EDG Receptors

S1P₁ (EDG-1) GTP [γ-³⁵S] binding assay: Homogenized membranes are prepared from CHO cell clones stably expressing a human EDG-1 N-terminal c-myc tag. Cells are grown in suspension in two 850 cm² roller bottles for three or fours days before harvesting. The cells are centrifuged down, washed once with cold PBS, and resuspended in ≦20 ml of Buffer A (20 mM HEPES, pH 7.4, 10 mM EDTA, EDTA-free complete protease inhibitor cocktail [1 tablet/25 ml]). The cell suspension is homogenized on ice, using a Polytron homogenizer at 30000 rpm at three intervals of 15 seconds each. The homogenate is first centrifuged at 2000 rpm on a tabletop low speed centrifuge for 10 minutes. The supernatant, after passing through a cell strainer, is then re-centrifuged at 50,000×g for 25 minutes at 4° C. The pellet is resuspended into buffer B (15% glycerol, 20 mM HEPES, pH 7.4, 0.1 mM EDTA, EDTA-free complete protease inhibitor cocktail [1 tablet/10 ml]). Protein concentration of the preparation is determined using the BCA Protein Assay kit (Pierce) using BSA as standard. The membranes are aliquoted and kept frozen at −80° C.

Solutions of test compounds ranging from 10 mM to 0.01 nM are prepared in DMSO. S1P is diluted in 4% BSA solution as positive controls. The desired amount of membrane preparation is diluted with ice-cold assay buffer (20 mM HEPES, pH 7.4, 100 mM NaCl, 10 mM MgCl₂, 0.1% Fatty acid-free BSA, 5 □M GDP) and vortexed well. 2 μl or less of compound is distributed into each well of a round-bottom 96-well polystyrene assay plate, followed by addition of 100 □l of diluted membranes (3-10 μg/well) and kept on ice until the addition of hot GTPγS. [³⁵S]-GTPγS is diluted 1:1000 (v/v) with cold assay buffer and 100 μl is added into each well. The reaction is carried out at room temperature for 90 minutes before the membranes are harvested onto Perkin-Elmer Unifilter® GF/B-96 filter plate using a Packard Filtermate Harvester. After several washes with wash buffer (20 mM HEPES, pH 7.4, 100 mM NaCl, 10 mM MgCl₂₎, and a rinse with 95% ethanol, the filter is dried in a 37° C. oven for 30 minutes. MicroScint-20 is added and the plate sealed for scintillation counting on TopCount. EC50 values are obtained by fitting the GTP [γ-³⁵S] binding curves (raw data) with the dose response curve-fitting tool of GraphPad Prism. Six or twelve different concentrations are used to generate a concentration response curve (using three data points per concentration).

S1P3,-5,-6 and -8 GTP [γ-³⁵] binding assays are carried out in a comparable manner to the S1P1 GTP [γ-³⁵ ] binding assay using membranes from CHO cells stably expressing c-terminal c-myc tagged or untagged receptors. For each membrane preparation, titration experiments are first run with S1P control to determine the optimal amount of membranes to be added per assay well.

Compounds of formula I are tested according to the above assay and show binding affinity to S1P receptors, e.g. S1P1 receptors with an EC50<1 μM.

More particularly compounds of formula I may exhibit selectivity for the S1P1 receptor compared to S1P3, S1P4 and S1P5, e.g. may at least be 20 fold selective for S1P1 compared to S1P3, S1P4 and S1P5.

A.2 FLIPR Calcium Flux Assay

Compounds of the invention are tested for agonist activity on S1P1, S1P3, S1P5, and S1P6 with a FLIPR calcium flux assay. Briefly, CHO cells expressing an S1P receptor are maintained in F-12K medium (ATCC), containing 5% FBS, with 500 ug/ml of G418. Prior to the assay, the cells are plated in 384 black clear bottom plates at the density of 10,000 cells/well/25 □l in the medium of F-12K containing 1% FBS. The second day, the cells are washed three times (25 μl/each) with washing buffer. About 25 μl of dye are added to each well and incubated for 1 hour at 37° C. and 5% CO₂. The cells are then washed four times with washing buffer (25 μl/each). The calcium flux is assayed after adding 25 μl of SEW2871 (published by Rosen et al., used as reference) solution to each well of cells. The same assay is performed with cells expressing each of the different S1P receptors. Titration in the FLIPR calcium flux assay is recorded over a 3-minute interval, and quantitated as maximal peak height percentage response relative to S1P-1 activation. The compounds of the invention are active in this assay at a concentration of from 10⁻¹² and 3.10⁻⁵ nM.

For example example 1 has an EC₅₀=8.7 nM for S1P-1 and an EC₅₀>1 um for all the other isoforms (S1P-2, S1P-3, S1P-4, S1P-5).

B. In vivo: Screening Assays for Measurement of Blood Lymphocyte Depletion

Measurement of circulating lymphocytes: Compounds to be tested are dissolved in DMSO/PEG200 and further diluted with deionized water. Rats (Lewis strain, female, 6-12 weeks old) are administered 1 mg/kg of compound to be tested in 4 ml/kg vehicle (max. 2% DMSO/max. 2% PEG200/water) via per os application. DMSO/PEG200/water and FTY720 (0.3 mg/kg) are included as negative and positive controls, respectively.

Blood is collected from the sublingual vein 2, 6, 24 and 48 hours after administration under short isoflurane anesthesia. Whole blood samples are subjected to hematology analysis. Peripheral lymphocyte counts are determined using an automated analyzer. Subpopulations of peripheral blood lymphocytes are stained by fluorochrome-conjugated specific antibodies and analyzed using a fluorescent activating cell sorter (Facscalibur). Two rats are used to assess the lymphocyte depletion activity of each compound screened. The result is an ED₅₀, which is defined as the effective dose required to display 50% of blood lymphocyte depletion. Compounds of formula I are tested according to the above assay and have an ED₅₀ of less than 10 mg/kg. For example compound of example 19 has an ED₅₀=0.5 mg/kg at 6 hours.

The compounds of formula I are, therefore, useful in the treatment and/or prevention of diseases or disorders mediated by lymphocytes interactions, e.g. in transplantation, such as acute or chronic rejection of cell, tissue or organ allo- or xenografts or delayed graft function, graft versus host disease, autoimmune diseases, e.g. rheumatoid arthritis, systemic lupus erythematosus, hashimoto's thyroidis, multiple sclerosis, myasthenia gravis, diabetes type I or II and the disorders associated therewith, vasculitis, pernicious anemia, Sjoegren syndrome, uveitis, psoriasis, Graves ophthalmopathy, alopecia areata and others, allergic diseases, e.g. allergic asthma, atopic dermatitis, allergic rhinitis/conjunctivitis, allergic contact dermatitis, inflammatory diseases optionally with underlying aberrant reactions, e.g. inflammatory bowel disease, Crohn's disease or ulcerative colitis, intrinsic asthma, inflammatory lung injury, inflammatory liver injury, inflammatory glomerular injury, atherosclerosis, osteoarthritis, irritant contact dermatitis and further eczematous dermatitises, seborrhoeic dermatitis, cutaneous manifestations of immunologically-mediated disorders, inflammatory eye disease, keratoconjunctivitis, myocarditis or hepatitis, ischemia/reperfusion injury, e.g. myocardial infarction, stroke, gut ischemia, renal failure or hemorrhage shock, traumatic shock, cancer, e.g. breast cancer, T cell lymphomas or T cell leukemias, infectious diseases, e.g. toxic shock (e.g. superantigen induced), septic shock, adult respiratory distress syndrome or viral infections, e.g. AIDS, viral hepatitis, chronic bacterial infection, or senile dementia. Examples of cell, tissue or solid organ transplants include e.g. pancreatic islets, stem cells, bone marrow, corneal tissue, neuronal tissue, heart, lung, combined heart-lung, kidney, liver, bowel, pancreas, trachea or oesophagus. For the above uses the required dosage will of course vary depending on the mode of administration, the particular condition to be treated and the effect desired.

In general, satisfactory results are indicated to be obtained systemically at daily dosages of from about 0.03 to 5.0 mg/kg per body weight. An indicated daily dosage in the larger mammal, e.g. humans, is in the range from about 0.5 mg to about 500 mg, conveniently administered, for example, in divided doses up to four times a day or in retard form. Suitable unit dosage forms for oral administration comprise from ca. 0.1 to 50 mg active ingredient.

The compounds of formula I may be administered by any conventional route, in particular enterally, e.g. orally, e.g. in the form of tablets or capsules, or parenterally, e.g. in the form of injectable solutions or suspensions, topically, e.g. in the form of lotions, gels, ointments or creams, or in a nasal or a suppository form. Pharmaceutical compositions comprising a compound of formula I in free form or in pharmaceutically acceptable salt form in association with at least one pharmaceutical acceptable carrier or diluent may be manufactured in conventional manner by mixing with a pharmaceutically acceptable carrier or diluent.

The compounds of formula I may be administered in free form or in pharmaceutically acceptable salt form e.g. as indicated above. Such salts may be prepared in conventional manner and exhibit the same order of activity as the free compounds.

In accordance with the foregoing the present invention further provides:
1.1 A method for preventing or treating disorders or diseases mediated by lymphocytes, e.g. such as indicated above, in a subject in need of such treatment, which method comprises administering to said subject an effective amount of a compound of formula I or a pharmaceutically acceptable salt thereof;
1.2 A method for preventing or treating acute or chronic transplant rejection or T-cell mediated inflammatory or autoimmune diseases, e.g. as indicated above, in a subject in need of such treatment, which method comprises administering to said subject an effective amount of a compound of formula I or a pharmaceutically acceptable salt thereof;
2. A compound of formula I, in free form or in a pharmaceutically acceptable salt form for use as a pharmaceutical, e.g. in any of the methods as indicated under 1.1 or 1.2 above.
3. A pharmaceutical composition, e.g. for use in any of the methods as in 1.1 or 1.2 above comprising a compound of formula I in free form or pharmaceutically acceptable salt form in association with a pharmaceutically acceptable diluent or carrier therefor.
4. A compound of formula I or a pharmaceutically acceptable salt thereof for use in the preparation of a pharmaceutical composition for use in any of the method as in 1.1 or 1.2 above.

The compounds of formula I may be administered as the sole active ingredient or in conjunction with, e.g. as an adjuvant to, other drugs e.g. immunosuppressive or immunomodulating agents or other anti-inflammatory agents, e.g. for the treatment or prevention of allo- or xenograft acute or chronic rejection or inflammatory or autoimmune disorders, or a chemotherapeutic agent, e.g a malignant cell anti-proliferative agent. For example, the compounds of formula I may be used in combination with a calcineurin inhibitor, e.g. cyclosporin A or FK 506; a mTOR inhibitor, e.g. rapamycin, 40-O-(2-hydroxyethyl)-rapamycin, CCI779, ABT578, AP23573, biolimus-7 or biolimus-9; an ascomycin having immuno-suppressive properties, e.g. ABT-281, ASM981, etc.; corticosteroids; cyclophosphamide; azathioprene; methotrexate; leflunomide; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a JAK3 kinase inhibitor, e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo-4+-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin4-yl)-amino]-piperidin-1-yl}-3-oxo-propionitrile, in free form or in a pharmaceutically acceptable salt form, e.g. mono-citrate (also called CP-690,550), or a compound as disclosed in WO 04/052359 or WO 05/066156; a S1P receptor agonist or modulator, e.g. FTY720 optionally phosphorylated or an analog thereof, e.g. 2-amino-2-[4-(3-benzyloxyphenylthio)-2-chlorophenyl]ethyl-1,3-propanediol optionally phosphorylated or 1-{4-[1-(4-cyclohexyl-3-trifluoromethyl-benzyloxyimino)-ethyl]-2-ethyl-benzyl}-azetidine-3-carboxylic acid or its pharmaceutically acceptable salts; immunosuppressive monoclonal antibodies, e.g., monoclonal antibodies to leukocyte receptors, e.g., MHC, CD2, CD3, CD4, CD7, CD8, CD25, CD28, CD40, CD45, CD52, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM4 antagonists or VLA-4 antagonists; or a chemotherapeutic agent, e.g. paclitaxel, gemcitabine, cisplatinum, doxorubicin or 5-fluorouracil; or an anti-infectious agent.

Where the compounds of formula I are administered in conjunction with other immunosuppressive/immunomodulatory, anti-inflammatory. chemotherapeutic or anti-infectious therapy, dosages of the co-administered immunosuppressant, immunomodulatory, anti-inflammatory, chemotherapeutic or anti-infectious compound will of course vary depending on the type of co-drug employed, e.g. whether it is a steroid or a calcineurin inhibitor, on the specific drug employed, on the condition being treated and so forth. In accordance with the foregoing the present invention provides in a yet further aspect:
5. A method as defined above comprising co-administration, e.g. concomitantly or in sequence, of a therapeutically effective non-toxic amount of a compound of formula I and at least a second drug substance, e.g. an immunosuppressant, immunomodulatory, anti-inflammatory or chemotherapeutic drug, e.g. as indicated above.
6. A pharmaceutical combination, e.g. a kit, comprising a) a first agent which is a compound of formula I as disclosed herein, in free form or in pharmaceutically acceptable salt form, and b) at least one co-agent, e.g. an immunosuppressant, immunomodulatory, anti-inflammatory, chemotherapeutic or anti-infectious agent. The kit may comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula I and a co-agent, are both administered to a patient simultaneously in the form of a single entity or dosage. The term “non-fixed combination” means that the active ingredients, e.g. a compound of formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

1. A compound of formula I 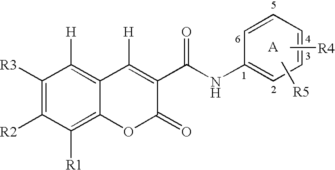
wherein
each of R₁ and R₂, independently, is selected from the group consisting of hydrogen; halogen; nitro; optionally substituted C₁₋₈alkyl; optionally substituted haloC₁₋₈alkyl; optionally substituted C₁₋₈alkyl-carbonyl; optionally substituted C₁₋₈alkenyl; optionally substituted C₁₋₈alkoxy; optionally substituted haloC₁₋₈alkoxy; C₁₋₈alkynyl; optionally substituted C₃₋₆cycloalkyl; optionally substituted C₃₋₈cycloalkyl-oxy; heterocyclic residue; optionally substituted aryl;
or R₁ and R₂ form together an optionally substituted C₃₋₈cycloalkyl or a heterocyclic residue;
R₃ is hydrogen; halogen; optionally substituted C₁₋₈alkyl; optionally substituted C₁₋₈alkoxy; optionally substituted haloC₁₋₈alkoxy; C₁₋₈alkenyl;
R₄ is of formula C₁₋₂alkyl-NR_{c}R_{d} wherein
the C₁₋₂alkyl is optionally substituted or is substituted by two alkyl residues on the same C atom wherein the two alkyl residues optionally form together with the C atom to which they are bound a C₃₋₈cycloalkyl;
each of R_{c} and R_{d}, independently, is selected from the group consisting of hydrogen; optionally substituted C₁₋₈alkyl; haloC₁₋₈alkyl; C₃₋₆cycloalkyl; C₁₋₈alkylcarbonyl; C₁₋₆alkoxycarbonyl; and C₁₋₆alkyne; or
R_{c} and R_{d} form together with the nitrogen atom to which they are bound an optionally substituted heterocyclic residue;
and R₄ is in position 3 or 4;
R₅ is hydrogen; hydroxyl; halogen; haloC₁₋₈alkyl; optionally substituted C₁₋₈alkyl; C₁₋₈alkoxy; or haloC₁₋₈alkoxy;
and R₅ is in position 2 or 3;
or R₄ and R₅ are in position 4 and 3, respectively, and form together a heterocyclic residue;
Ring A comprises no heteroatom or one or two ring heteroatom;
with the proviso that R₁ and R₂ are not both hydrogen;
or a physiologically hydrolysable derivative thereof, a salt, hydrate and/or solvate thereof.

2. A compound according to claim 1 wherein each of R₁ and R₂, independently, is hydrogen; optionally substituted C₁₋₈alkyl; optionally substituted haloC₁₋₈alkyl; optionally substituted C₁₋₈alkoxy; or optionally substituted haloC₁₋₈alkoxy;
or a physiologically hydrolysable derivative thereof, a salt, hydrate and/or solvate thereof.

3. A compound according to claim 1 wherein R₄ is of formula C₁₋₂alkyl-NR_{c}R_{d} wherein the C₁₋₂alkyl is optionally substituted or is substituted by two alkyl residues on the same C atom wherein the two alkyl residues optionally form together with the C atom to which they are bound a C₃₋₈cycloalkyl;
each of R_{c} and R_{d}, independently, is selected from the group consisting of hydrogen;
optionally substituted C₁₋₈alkyl; haloC₁₋₈alkyl; or R_{c} and R_{d} form together with the nitrogen atom to which they are bound an optionally substituted heterocyclic residue;
or a physiologically hydrolysable derivative thereof, a salt, hydrate and/or solvate thereof.

4. A compound according to claim 1 wherein R₃ and R₅ are hydrogen.

5. A compound according to claim 1 which is selected from
N-[4-(aminomethyl)phenyl]-7-methoxy-2-oxo-8-propyl-2H-chromene-3-carboxamide;
7-(2-fluoroethoxy)-N-{4-[(methylamino)methyl]phenyl}-2-oxo-8-propyl-2H-chromene-3-carboxamide;
N-[4-(aminomethyl)-2-(trifluoromethyl)phenyl]-7-ethoxy-2-oxo-8-propyl-2H-chromen-3-carboxamide;
N-[4-(aminomethyl)-3-methylphenyl]-7-methoxy-2-oxo-8-propyl-2H-chromene-3-carboxamide;
N-[4-(aminomethyl)-3-(trifluoromethyl)phenyl]-7-ethoxy-2-oxo-8-propyl-2H-chromene-3-carboxamide;
7-ethoxy-N-{4-[(methylamino)methyl]phenyl}-2-oxo-8-propyl-2H-chromene-3-carboxamide;
N-[4-(aminomethyl)-3-chlorophenyl]-7-methoxy-2-oxo-8-propyl-2H-chromene-3-carboxamide;
N-[4-(aminomethyl)-3-chlorophenyl]-7-ethoxy-2-oxo-8-propyl-2H-chromene-3-carboxamide;
N-[4-(aminomethyl)phenyl]-7-ethoxy-2-oxo-8-propyl-2H-chromene-3-carboxamide;
N-(4-{[(8-allyl-7-methoxy-2-oxo-2H-chromen-3-yl)carbonyl]amino}benzyl)-beta-alanine;
N-[4-(aminomethyl)-2-methoxyphenyl]-7-methoxy-2-oxo-8-propyl-2H-chromene-3-carboxamide;
N-[4-(aminomethyl)-2-methylphenyl]-7-methoxy-2-oxo-8-propyl-2H-chromene-3-carboxamide;
N-[4-(aminomethyl)-2-(trifluoromethyl)phenyl]-7-methoxy-2-oxo-8-propyl-2H-chromene-3-carboxamide;
N-[4-(aminomethyl)-3-(trifluoromethyl)phenyl]-7-methoxy-2-oxo-8-propyl-2H-chromene-3-carboxamide;
N-[4-(aminomethyl)-2-chlorophenyl]-7-methoxy-2-oxo-8-propyl-2H-chromene-3-carboxamide;
N-[4-(1-amino-1-methylethyl)phenyl]-7-ethoxy-2-oxo-8-propyl-2H-chromene-3-carboxamide;
N-[4-(aminomethyl)-2-(trifluoromethoxy)phenyl]-7-methoxy-2-oxo-8-propyl-2H-chromene-3-carboxamide;
7-{[(1R)-1-methylpropyl]oxy}-2-oxo-8-propyl-N-[4-(pyrrolidin-1-ylmethyl)phenyl]-2H-chromene-3-carboxamide;
N-[4-(1-aminocyclopropyl)phenyl]-7-methoxy-2-oxo-8-propyl-2H-chromene-3-carboxamide;
N-[4-(1-aminocyclopropyl)phenyl]-7-ethoxy-2-oxo-8-propyl-2H-chromene-3-carboxamide;
N-[4-(1-amino-1-methylethyl)phenyl]-7-methoxy-2-oxo-8-propyl-2H-chromene-3-carboxamide;
N-{4-[(dimethylamino)methyl]phenyl}-7-ethoxy-2-oxo-8-propyl-2H-chromene-3-carboxamide;
7-ethoxy-2-oxo-8-propyl-N-[4-(pyrrolidin-1-ylmethyl)phenyl]-2H-chromene-3-carboxamide;
N-[4-(aminomethyl)-2-(trifluoromethoxy)phenyl]-8-isopropoxy-7-methoxy-2-oxo-2H-chromene-3-carboxamide;
N-[4-(aminomethyl)-2-(trifluoromethoxy)phenyl]-7-ethoxy-2-oxo-8-propyl-2H-chromene-3-carboxamide;
N-{4-[(ethylamino)methyl]phenyl}-7-methoxy-2-oxo-8-propyl-2H-chromene-3-carboxamide;
7-methoxy-N-{4-[(methylamino)methyl]phenyl}-2-oxo-8-propyl-2H-chromene-3-carboxamide;
N-(4-{[(2-hydroxyethyl)amino]methyl}phenyl)-7-methoxy-2-oxo-8-propyl-2H-chromene-3-carboxamide;
N-{4-[(cyclopropylamino)methyl]phenyl}-7-methoxy-2-oxo-8-propyl-2H-chromene-3-carboxamide;
7-methoxy-N-{4-[1-methyl-1-(methylamino)ethyl]phenyl}-2-oxo-8-propyl-2H-chromene-3-carboxamide;
methyl N-(4-{[(8-allyl-7-methoxy-2-oxo-2H-chromen-3-yl)carbonyl]amino}benzyl)-beta-alaninate;
N-[4-(aminomethyl)phenyl]-7-methoxy-2-oxo-8-propoxy-2H-chromene-3-carboxamide;
N-(4-{[(2-hydroxyethyl)(methyl)amino]methyl}phenyl)-7-methoxy-2-oxo-8-propyl-2H-chromene-3-carboxamide;
N-{4-[(cyclobutylamino)methyl]phenyl}-7-methoxy-2-oxo-8-propyl-2H-chromene-3-carboxamide;
7-methoxy-2-oxo-8-propyl-N-[4-(pyrrolidin-1-ylmethyl)phenyl]-2H-chromene-3-carboxamide;
8-allyl-N-{4-[(dimethylamino)methyl]phenyl}-7-methoxy-2-oxo-2H-chromene-3-carboxamide;
N-{4-[(dimethylamino)methyl]phenyl}-7-methoxy-2-oxo-8-propyl-2H-chromene-3-carboxamide;
N-[4-(aminomethyl)phenyl]-7-methyl-2-oxo-8-propyl-2H-chromene-3-carboxamide;
7-methoxy-N-{4-[1-(methylamino)cyclopropyl]phenyl}-2-oxo-8-propyl-2H-chromene-3-carboxamide;
7-methoxy-N-[4-(morpholin-4-ylmethyl)-2-(trifluoromethoxy)phenyl]-2-oxo-8-propyl-2H-chromene-3-carboxamide;
8-ally-7-methoxy-2-oxo-N-[4-(piperidin-1-ylmethyl)phenyl]-2H-chromene-3-carboxamide;
N-{4-[(benzylamino)methyl]phenyl}-7-methoxy-2-oxo-8-propyl-2H-chromene-3-carboxamide;
N-[4-(aminomethyl)phenyl]-8-isopropoxy-7-methoxy-2-oxo-2H-chromene-3-carboxamide;
7-methoxy-2-oxo-N-[4-(piperidin-1-ylmethyl)phenyl]-8-propyl-2H-chromene-3-carboxamide;
N-[4-(aminomethyl)phenyl]-8-ethoxy-7-methoxy-2-oxo-2H-chromene-3-carboxamide;
N-[4-(2-aminoethyl)phenyl]-7-methoxy-2-oxo-8-propyl-2H-chromene-3-carboxamide;
N-[4-(aminomethyl)-3-methoxyphenyl]-7-methoxy-2-oxo-8-propyl-2H-chromene-3-carboxamide;
tert-butyl N-(4-{[(8-allyl-7-methoxy-2-oxo-2H-chromen-3-yl)carbonyl]amino}benzyl)-beta-alaninate;
N-{4-[11-(dimethylamino)-1-methylethyl]phenyl}-7-methoxy-2-oxo-8-propyl-2H-chromene-3-carboxamide;
N-[4-(aminomethyl)phenyl]-6-chloro-7-methoxy-2-oxo-8-propyl-2H-chromene-3-carboxamide;
N-[4-(aminomethyl)phenyl]-7,8-diisopropoxy-2-oxo-2H-chromene-3-carboxamide:
N-[4-(aminomethyl)-2-hydroxyphenyl]-7-methoxy-2-oxo-8-propyl-2H-chromene-3-carboxamide;
N-[4-(aminomethyl)phenyl]-6,8-di-tert-butyl-2-oxo-2H-chromene-3-carboxamide;
4-{[(7-methoxy-2-oxo-8-propyl-2H-chromen-3-yl)carbonyl]amino}-L-phenylalanine;
N-{4-[(isopropylamino)methyl]phenyl}-7-methoxy-2-oxo-8-propyl-2H-chromene-3-carboxamide;
7-methoxy-N-(4-{[(2-methoxyethyl)amino]methyl}phenyl)-2-oxo-8-propyl-2H-chromene-3-carboxamide;
N-(4-{[(8-allyl-7-methoxy-2-oxo-2H-chromen-3-yl)carbonyl]amino}benzoyl)-beta-alanine;
6,8-di-tert-butyl-2-oxo-N-[4-(pyrrolidin-1-ylmethyl)phenyl]2H-chromene-3-carboxamide;
N-{4-[(2R)-2-aminopropyl]phenyl}-7-methoxy-2-oxo-8-propyl-2H-chromene-3-carboxamide;
N-[4-(aminomethyl)-3-hydroxyphenyl]-7-methoxy-2-oxo-8-propyl-2H-chromene-3-carboxamide;
N-[4-(acetamidomethyl)phenyl]-8-allyl-7-methoxy-2-oxo-2H-chromene-3-carboxamide;
N-[4-(aminomethyl)-2-(trifluoromethoxy)phenyl]-6,8-di-tert-butyl-2-oxo-2H-chromene-3-carboxamide;
N-{4-[(dimethylamino)methyl]-2-(trifluoromethoxy)phenyl}-7-methoxy-2-oxo-8-propyl-2H-chromene-3-carboxamide;
7-ethoxy-N-[4-(morpholin-4-ylmethyl)phenyl]-2-oxo-8-propyl-2H-chromene-3-carboxamide;
7-methoxy-N-(4-{[methyl(prop-2-yn-1-yl)amino]methyl}phenyl)-2-oxo-8-propyl-2H-chromene-3-carboxamide;
methyl N-(4-{[(8-allyl-2-oxo-2H-chromen-3-yl)carbonyl]amino}benzyl)-beta-alaninate;
N-[4-(aminomethyl)phenyl]-7,8-dimethoxy-2-oxo-2H-chromene-3-carboxamide;
7-methoxy-N-[4-(morpholin-4-ylmethyl)phenyl]-2-oxo-8-propyl-2H-chromene-3-carboxamide;
N-[4-({[2-(dimethylamino)-2-oxoethyl]amino}methyl)phenyl]-7-methoxy-2-oxo-8-propyl-2H-chromene-3-carboxamide;
N-{4-[(tert-butylamino)methyl]phenyl}-7-methoxy-2-oxo-8-propyl-2H-chromene-3-carboxamide;
8-allyl-7-methoxy-N-[4-(morpholin-4-ylmethyl)phenyl]-2-oxo-2H-chromene-3-carboxamide;
N-(4-{[bis(2-methoxyethyl)amino]methyl}phenyl)-7-methoxy-2-oxo-8-propyl-2H-chromene-3-carboxamide;
7-methoxy-N-{3-[(methylamino)methyl]phenyl}-2-oxo-8-propyl-2H-chromene-3-carboxamide;
N-(4-{[(2R,6S)-2,6-dimethylmorpholin-4-yl]methyl}phenyl)-7-methoxy-2-oxo-8-propyl-2H-chromene-3-carboxamide;
N-(4-[(dimethylamino)methyl]phenyl)-8-methyl-2-oxo-6-(trifluoromethoxy)-2H-chromene-3-carboxamide;
N-[4-(morpholin-4-ylmethyl)phenyl]-2-oxo-8-propyl-7-(trifluoromethyl)-2H-chromene-3-carboxamide;
7-methoxy-N-[4-(1-methyl-1-morpholin-4-ylethyl)phenyl]-2-oxo-8-propyl-2H-chromene-3-carboxamide;
8-allyl-7-methoxy-2-oxo-N-{4-[(2-oxopyrrolidin-1-yl)methyl]phenyl}-2H-chromene-3-carboxamide;
7-methoxy-N-[4-(1-morpholin-4-ylethyl)phenyl]-2-oxo-8-propyl-2H-chromene-3-carboxamide;
N-[3-(2-aminoethyl)phenyl]-7-methoxy-2-oxo-8-propyl-2H-chromene-3-carboxamide;
6-allyl-N-{4-[(dimethylamino)methyl]phenyl}-methoxy-2-oxo-2H-chromene-3-carboxamide;
N-{4-[1-(dimethylamino)cyclopropyl]phenyl}-7-methoxy-2-oxo-8-propyl-2H-chromene-3-carboxamide;
7-methoxy-N-{4-[(4-methylpiperazin-1-yl)methyl]phenyl}-2-oxo-8-propyl-2H-chromene-3-carboxamide;
7-methoxy-2-oxo-8-propyl-N-(4-{[(3,3,3-trifluoropropyl)amino]methyl}phenyl)-2H-chromene-3-carboxamide;
7-methoxy-N-{4-[(methylamino)methyl]phenyl}-2-oxo-2H-chromene-3-carboxamide;
8-allyl-N-{4-[(dimethylamino)methyl]phenyl}-2-oxo-2H-chromene-3-carboxamide;
N-{4-[(E)-(dimethylhydrazono)methyl]phenyl}-7-methoxy-2-oxo-8-propyl-2H-chromene-3-carboxamide;
7-methoxy-2-oxo-N-[3-(piperazin-1-ylmethyl)phenyl]-8-propyl-2H-chromene-3-carboxamide;
N-{4-[(1E)-N-hydroxyethanimidoyl]phenyl}-7-methoxy-2-oxo-8-propyl-2H-chromene-3-carboxamide;
tert-butyl (4-{[(8-allyl-7-methoxy-2-oxo-2H-chromen-3-yl)carbonyl]amino}benzyl)carbamate;
N-[4-(aminomethyl)phenyl]-7-methoxy-2-oxo-2H-chromene-3-carboxamide;
7-methoxy-N-{4-[(methylamino)methyl]-2-(trifluoromethoxy)phenyl}-2-oxo-8-propyl-2H-chromene-3-carboxamide;
N-{4-[(1S,2S)-2-(dimethylamino)-1,3-dihydroxypropyl]phenyl}-7-methoxy-2-oxo-8-propyl-2H-chromene-3-carboxamide;
N-{4-[(1R,2R)-2-amino-1,3-dihydroxypropyl]phenyl}-7-methoxy-2-oxo-8-propyl-2H-chromene-3-carboxamide;
N-{4-[(1S,2S)-2-amino-1,3-dihydroxypropyl]phenyl}-7-methoxy-2-oxo-8-propyl-2H-chromene-3-carboxamide;
7-methoxy-N-{4-[(4-oxidomorpholin-4-yl)methyl]phenyl}-2-oxo-8-propyl-2H-chromene-3-carboxamide;
N-[4-(aminomethyl)phenyl]-7-{[(1R)-1-methylpropyl]oxy}-2-oxo-8-propyl-2H-chromene-3-carboxamide;
N-[4-(aminomethyl)phenyl]-7-{[(1S)-1-methylpropyl]oxy}-2-oxo-8-propyl-2H-chromene-3-carboxamide;
N-[4-(aminomethyl)phenyl]-7-(2-fluoroethoxy)-2-oxo-8-propyl-2H-chromene-3-carboxamide;
N-[4-(aminomethyl)phenyl]-7-(2,2-difluoroethoxy)-2-oxo-8-propyl-2H-chromene-3-carboxamide;
N-[4-(aminomethyl)phenyl]-7-isopropoxy-2-oxo-8-propyl-2H-chromene-3-carboxamide;
N-[4-(1-amino-1-methylethyl)phenyl]-7-(2,2-difluoroethoxy)-2-oxo-8-propyl-2H-chromene-3-carboxamide;
N-{4-[(methylamino)methyl]phenyl}-7-{[(1R)-1-methylpropyl]oxy}-2-oxo-8-propyl-2H-chromene-3-carboxamide;
N-{4-[(methylamino)methyl]phenyl}-7-{[(1S)-1-methylpropyl]oxy}-2-oxo-8-propyl-2H-chromene-3-carboxamide;
N-[4-(aminomethyl)phenyl]-2-oxo-7-propoxy-8-propyl-2H-chromene-3-carboxamide;
N-[4-(aminomethyl)-2-(trifluoromethoxy)phenyl]-7-isopropoxy-2-oxo-8-propyl-2H-chromene-3-carboxamide;
7-isopropoxy-N-{4-[(methylamino)methyl]phenyl}-2-oxo-8-propyl-2H-chromene-3-carboxamide;
N-[4-(aminomethyl)phenyl]-7-isobutoxy-2-oxo-8-propyl-2H-chromene-3-carboxamide;
N-{4-[(dimethylamino)methyl]phenyl}-7-{[(1R)-1-methylpropyl]oxy}-2-oxo-8-propyl-2H-chromene-3-carboxamide;
N-[4-(1-amino-1-methylethyl)phenyl]-7-isopropoxy-2-oxo-8-propyl-2H-chromene-3-carboxamide;
7-{[(1S)-1-methylpropyl]oxy}-2-oxo-8-propyl-N-[4-(pyrrolidin-1-ylmethyl)phenyl]-2H-chromene-3-carboxamide;
N-[4-(1-amino-1-methylethyl)phenyl]-7-{[(1S)-1-methylpropyl]oxy}-2-oxo-8-propyl-2H-chromene-3-carboxamide;
N-[4-(aminomethyl)phenyl]-7-(cyclopropylmethoxy)-2-oxo-8-propyl-2H-chromene-3-carboxamide;
7-(cyclobutyloxy)-N-{4-[(dimethylamino)methyl]phenyl}-2-oxo-8propyl-2H-chromene-3-carboxamide;
7-(2,2-difluoroethoxy)-N-{4-[(methylamino)methyl]phenyl}-2-oxo-8-propyl-2H-chromene-3-carboxamide;
N-[4-(aminomethyl)phenyl]-7-(cyclopentyloxy)-2-oxo-8-propyl-2H-chromene-3-carboxamide;
7-(1-cyclopropylethoxy)-N-{4-[(dimethylamino)methyl]phenyl}-2-oxo-8-propyl-2H-chromene-3-carboxamide;
N-{4-[(dimethylamino)methyl]phenyl}-7-{[(1S)-1-methylpropyl]oxy}-2-oxo-8-propyl-2H-chromene-3-carboxamide;
7-(2-fluoroethoxy)-2-oxo-8-propyl-N-[4-(pyrrolidin-1-ylmethyl)phenyl]-2H-chromene-3-carboxamide;
N-{4-[(dimethylamino)methyl]phenyl}-7-isopropoxy-2-oxo-8-propyl-2H-chromene-3-carboxamide;
7-(cyclopropylmethoxy)-N-{4-[(dimethylamino)methyl]phenyl}-2-oxo-8-propyl-2H-chromene-3-carboxamide;
N-{4-[(dimethylamino)methyl]phenyl}-7-(1,2-dimethylpropoxy)-2-oxo-8-propyl-2H-chromene-3-carboxamide;
N-(4-[(dimethylamino)methyl]phenyl}-7-isobutoxy-2-oxo-8-propyl-2H-chromene-3-carboxamide;
7-{[(1( S)-1-methylpropyl]oxy}-N-[4-(morpholin-4-ylmethyl)phenyl]-2-oxo-8-propyl-2H-chromene-3-carboxamide;
7-(cyclopentyloxy)-N-{4-[(dimethylamino)methyl]phenyl}-2-oxo-8-propyl-2H-chromene-3-carboxamide;
7-isopropoxy-N-[4-(morpholin-4-ylmethyl)phenyl]-2-oxo-8-propyl-2H-chromene-3-carboxamide;
7-isobutoxy-N-[4-(morpholin-4-ylmethyl)phenyl]-2-oxo-8-propyl-2H-chromene-3-carboxamide;
7-(cyclopentyloxy)-N-[4-(morpholin-4-ylmethyl)phenyl]-2-oxo-8-propyl-2H-chromene-3-carboxamide;
N-[4-(1-aminocyclopropyl)phenyl]-7-ethoxy-8-ethyl-2-oxo-2H-chromene-3-carboxamide;
N-[4-(1-amino-1-methylethyl)phenyl]-7-ethoxy-8-ethyl-2-oxo-2H-chromene-3-carboxamide;
8-butyl-7-methoxy-N-{4-[(methylamino)methyl]phenyl}-2-oxo-2H-chromene-3-carboxamide;
N-{4-[(dimethylamino)methyl]phenyl}-8-ethyl-7-methoxy-2-oxo-2H-chromene-3-carboxamide;
8-ethyl-7-methoxy-N-{4-[(methylamino)methyl]phenyl}-2-oxo-2H-chromene-3-carboxamide;
N-(4-{[cyclopropyl(methyl)amino]methyl}phenyl)-7-methoxy-2-oxo-8-propyl-2H-chromene-3-carboxamide;
N-(4-{[cyclopropyl(methyl)amino]methyl}phenyl)-7-ethoxy-2-oxo-8-propyl-2H-chromene-3-carboxamide;
N-[4-(1-amino-1-methylethyl)phenyl]-7-methyl-2-oxo-8-propyl-2H-chromene-3-carboxamide;
N-{4-[(cyclopropylamino)methyl]phenyl}-7-ethoxy-2-oxo-8-propyl-2H-chromene-3-carboxamide;
N-[4-(1-aminocyclopropyl)phenyl]-7-methyl-2-oxo-8-propyl-2H-chromene-3-carboxamide;
N-[4-(2-amino-1-hydroxyethyl)phenyl]-7-methoxy-2-oxo-8-propyl-2H-chromene-3-carboxamide;
N-[4-(1-amino-1-methylethyl)phenyl]-7-methoxy-8-methyl-2-oxo-2H-chromene-3-carboxamide;
N-[4-(1-aminocyclopropyl)phenyl]-7-methoxy-8-methyl-2-oxo-2H-chromene-3-carboxamide;
N-{4-[(2S)-2-amino-3-hydroxypropyl]phenyl}-7-methoxy-2-oxo-propyl-2H-chromene-3-carboxamide;
N-[4-(1-aminocyclopropyl)phenyl]-2-oxo-8-propyl-7-(trifluoromethyl)-2H-chromene-3-carboxamide;
N-{4-[(2R)-2-amino-3-hydroxypropyl]phenyl}-7-methoxy-2-oxo-8-propyl-2H-chromene-3-carboxamide;
N-[4-(1-aminocyclopropyl)phenyl]-8-isopropoxy-7-methoxy-2-oxo-2H-chromene-3-carboxamide;
N-[4-(1-amino-1-methylethyl)phenyl]-8-isopropoxy-7-methoxy-2-oxo-2H-chromene-3-carboxamide;
N-[4-(aminomethyl)phenyl]-7-methoxy-8-methyl-2-oxo-2H-chromene-3-carboxamide;
N-[4-(aminomethyl)-2-(trifluoromethoxy)phenyl]-8-ethyl-7-methoxy-2-oxo-2H-chromene-3-carboxamide;
8-ethyl-7-methoxy-2-oxo-N-[4-(pyrrolidin-1-ylmethyl)phenyl]-2H-chromene-3-carboxamide;
8-ethyl-7-methoxy-N-[4-(morpholin-4-ylmethyl)phenyl]-2-oxo-2H-chromene-3-carboxamide;
8-ethyl-N-{4-[(isopropylamino)methyl]phenyl}-7-methoxy-2-oxo-2H-chromene-3-carboxamide;
8-ethyl-N-(4-{[ethyl(methyl)amino]methyl}phenyl)-7-methoxy-2-oxo-2H -chromene -3-carboxamide;
8-ethyl-N-{4-[(ethylamino)methyl]phenyl}-7-methoxy-2-oxo-2H-chromene-3-carboxamide;
N-[4-(1-amino-1-methylethyl)phenyl]-isopropyl-7-methoxy-2-oxo-2H-chromene-3-carboxamide;
N-[4-(1-aminocyclopropyl)phenyl]-8-isopropyl-7-methoxy-2-oxo-2H-chromene-3-carboxamide;
N-{4-[(1S)-1-aminoethyl]phenyl}-8-ethyl-7-methoxy-2-oxo-2H-chromene-3-carboxamide;
N-{4-[(1R)-1-aminoethyl]phenyl}-8-ethyl-7-methoxy-2-oxo-2H-chromene-3-carboxamide;
N-{4-[(1S,2S)-2-(dimethylamino)-1,3-dihydroxypropyl]phenyl}-8-ethyl-7-methoxy-2-oxo-2H-chromene-3-carboxamide,
N-{4-[(1S,2S)-2-amino-1,3-dihydroxypropyl]phenyl}-8-ethyl-7-methoxy-2-oxo-2H-chromene-3-carboxamide;
N-[4-(aminomethyl)-2-methoxyphenyl]-8-ethyl-7-methoxy-2-oxo-2H-chromene-3-carboxamide;
8-ethyl-N-(4-{[(3S)-3-hydroxypyrrolidin-1-yl]methyl}phenyl)-7-methoxy-2-oxo-2H-chromene-3-carboxamide;
8-ethyl-N-(4-{[(3R)-3-hydroxypyrrolidin-1-yl]methyl}phenyl)-7-methoxy-2-oxo-2H-chromene-3-carboxamide;
N-{4-[(cyclopropylamino)methyl]phenyl}-8-ethyl-7-methoxy-2-oxo-2H-chromene-3-carboxamide;
8-ethyl-N-(4-{[(2-hydroxyethyl)amino]methyl}phenyl)-7-methoxy-2-oxo-2H-chromene-3-carboxamide;
8-isopropyl-7-methoxy-N-{4-[(methylamino)methyl]phenyl}-2-oxo-2H-chromene-3-carboxamide;
8-isopropyl-7-methoxy-2-oxo-N-[4-(pyrrolidin-1-ylmethyl)phenyl]-2H-chromene-3-carboxamide;
N-(4-{[ethyl(methyl)amino]methyl}phenyl)-8-isopropyl-7-methoxy-2oxo-2H-chromene-3-carboxamide;
8-ethyl-N-(4-{[(2-hydroxyethyl)(methyl)amino]methyl}phenyl)-7-methoxy-2-oxo-2H-chromene-3-carboxamide;
N-{4-[(2R)-2-amino-3-hydroxypropyl]phenyl}-8-ethyl-7-methoxy-2-oxo-2H-chromene-3-carboxamide;
N-[4-(1-acetamidocyclopropyl)phenyl]-8-ethyl-7-methoxy-2-oxo-2H-chromene-3-carboxamide;
N-[4-(1-acetamidocyclopropyl)phenyl]-7-methoxy-2-oxo-8-propyl-2H-chromene-3-carboxamide;
7-(2,2-difluoroethoxy)-N-{4-[(dimethylamino)methyl]phenyl}-2-oxo-8propyl-2H-chromene-3-carboxamide;
7-(2,2-difluoroethoxy)-N-(4-{[(2-methoxyethyl)(methyl)amino]methyl}phenyl)-2-oxo-8-propyl-2H-chromene-3-carboxamide;
7-(2-fluoroethoxy)-N-(4-{[(2-methoxyethyl)(methyl)amino]methyl}phenyl)-2-oxo-8-propyl-2H-chromene-3-carboxamide;
7-(2,2-difluoroethoxy)-N-[4-(morpholin-4-ylmethyl)phenyl]-2-oxo-8-propyl-2H-chromene-3-carboxamide;
N-{4-[(dimethylamino)methyl]phenyl}-7-(2-fluoroethoxy)-2-oxo-8-propyl-2H-chromene-3-carboxamide;
7-(2-fluoroethoxy)-N-[4-(morpholin-4-ylmethyl)phenyl]-2-oxo-8-propyl-2H-chromene-3-carboxamide;
N-[4-(1-aminocyclopropyl)phenyl]-7-(2,2-difluoroethoxy)-2-oxo-8-propyl-2H-chromene-3-carboxamide;
N-[4-(1-aminocyclopropyl)phenyl]-7-(2-fluoroethoxy)-2-oxo-8-propyl-2H-chromene-3-carboxamide;
N-[4-(1-aminocyclopropyl)phenyl]-7-{[(1S)-1-methylpropyl]oxy}-2-oxo-8-propyl-2H-chromene-3-carboxamide;
N-[4-(1-amino-1-methylethyl)phenyl]-7-(2-fluoroethoxy)-2-oxo-8-propyl-2H-chromene-3-carboxamide;
N-{4-[(3,3-difluoropiperidin-1-yl)methyl]phenyl}-7-(2-fluoroethoxy)-2-oxo-8-propyl-2H-chromene-3-carboxamide;
7-(2,2-difluoroethoxy)-N-{4-[(3,3-difluoropiperidin-1-yl)methyl]phenyl}-2-oxo-8-propyl-2H-chromene-3-carboxamide;
N-{4-[(3,3-difluoropiperidin-1-yl)methyl]phenyl}-7-{[(1S)-1-methylpropyl]oxy}-2-oxo-8-propyl-2H-chromene-3-carboxamide;
N-{4-[(3,3-difluoropyrrolidin-1-yl)methyl]phenyl}-7-(2-fluoroethoxy)-2-oxo-8-propyl-2H-chromene-3-carboxamide;
7-(2,2-difluoroethoxy)-N-{4-[(3,3-difluoropyrrolidin-1-yl)methyl]phenyl}-2-oxo-8-propyl-2H-chromene-3-carboxamide;
N-{4-[(3,3-difluoropyrrolidin-1-yl)methyl]phenyl}-7-{[(1S)-1-methylpropyl]oxy}-2-oxo-8-propyl-2H-chromene-3-carboxamide;
N-(4-{[ethyl(methyl)amino]methyl}phenyl)-7-(2-fluoroethoxy)-2-oxo-8-propyl-2H-chromene-3-carboxamide;
7-(2,2-difluoroethoxy)-N-(4-{[ethyl(methyl)amino]methyl}phenyl)-2-oxo-8-propyl-2H-chromene-3-carboxamide;
N-[4-(aminomethyl)phenyl]-2-oxo-8-propyl-7-(3,3,3-trifluoropropoxy)-2H-chromene-3-carboxamide;
N-[4-(1-aminocyclopropyl)phenyl]-2-oxo-8-propyl-7-(3,3,3-trifluoropropoxy)-2H-chromene-3-carboxamide;
N-{4-[(dimethylamino)methyl]phenyl}-2-oxo-8-propyl-7-(3,3,3-trifluoropropoxy)-2H-chromene-3-carboxamide;
N-(4-{[cyclopropyl(methyl)amino]methyl}phenyl)-7-(2-fluoroethoxy)-2-oxo-8-propyl-2H-chromene-3-carboxamide;
N-{4-[(cyclopropylamino)methyl]phenyl}-7-(2-fluoroethoxy)-2-oxo-8-propyl-2H-chromene-3-carboxamide;
N-[4-(1-amino-1-methylethyl)phenyl]-2-oxo-8-propyl-7-(3,3,3-trifluoropropoxy)-2H-chromene-3-carboxamide;
7-methoxy-N-(4-{[(2-methoxyethyl)(methyl)amino]methyl}phenyl)-2-oxo-8-propyl-2H-chromene-3-carboxamide;
7-methoxy-N-[4-(morpholin-4-ylmethyl)-2-(trifluoromethyl)phenyl]-2-oxo-8-propyl-2H-chromene-3-carboxamide;
N-[2-chloro-4-(morpholin-4-ylmethyl)phenyl]-7-methoxy-2-oxo-8-propyl-2H-chromene-3-carboxamide;
7-ethoxy-N-(4-{[(2-methoxyethyl)(methyl)amino]methyl}phenyl)-2-oxo-8-propyl-2H-chromene-3-carboxamide;
8-allyl-N-{4-[(dimethylamino)methyl]phenyl}-7-ethyl-2-oxo-2H-chromene-3-carboxamide;
N-[4-(aminomethyl)phenyl]-8-ethyl-7-methoxy-2-oxo-2H-chromene-3-carboxamide;
7-methoxy-N-[2-methoxy-4-(morpholin-4-ylmethyl)phenyl]-2-oxo-8-propyl-2H-chromene-3-carboxamide;
N-[3-chloro-4-(morpholin-4-ylmethyl)phenyl]-7-methoxy-2-oxo-8-propyl-2H-chromene-3-carboxamide;
N-{4-[(dimethylamino)methyl]phenyl}-8-isopropyl-7-methoxy-2-oxo-2H-chromene-3-carboxamide;
4-{[(7-methoxy-2-oxo-8-propyl-2H-chromen-3-yl)carbonyl]amino}-D-phenylalanine;
N-{4-[(2R)-2,3-diamino-3-oxopropyl]phenyl}-7-methoxy-2-oxo-8-propyl-2H-chromene-3-carboxamide;
N-{4-[(2R)-2-amino-3-(dimethylamino)-3-oxopropyl]phenyl}-7-methoxy-2-oxo-8-propyl-2H-chromene-3-carboxamide;
8-allyl-N-{4-[(dimethylamino)methyl]phenyl}-7-isopropyl-2-oxo-2H-chromene-3-carboxamide;
N-{4-[(dimethylamino)methyl]phenyl}-7-ethyl-2-oxo-8-propyl-2H-chromene-3-carboxamide;
N-[4-(aminomethyl)phenyl]-8-butyl-7-methoxy-2-oxo-2H-chromene-3-carboxamide;
N-{4-[(3,3-difluoropiperidin-1-yl)methyl]phenyl}-7-ethoxy-2-oxo-8-propyl-2H-chromene-3-carboxamide;
N-{4-[(3,3-difluoropyrrolidin-1-yl)methyl]phenyl}-7-methoxy-2-oxo-8-propyl-2H-chromene-3-carboxamide;
N-{4-[(3,3-difluoropyrrolidin-1-yl)methyl]phenyl}-7-ethoxy-2-oxo-8-propyl-2H-chromene-3-carboxamide;
N-[4-(aminomethyl)phenyl]-7-ethyl-2-oxo-8-propyl-2H-chromene-3-carboxamide;
N-{4-[(dimethylamino)methyl]phenyl}-7-methoxy-8-(3-methylbutyl)-2-oxo-2H-chromene-3-carboxamide;
8-allyl-N-[4-(aminomethyl)phenyl]-7-methoxy-2-oxo-2H-chromene-3-carboxamide;
7-methoxy-8-(3-methylbutyl)-N-[4-(morpholin-4-ylmethyl)phenyl]-2-oxo-2H-chromene-3-carboxamide;
N-{4-[(dimethylamino)methyl]phenyl}-8-isobutyl-7-methoxy-2-oxo-2H-chromene-3-carboxamide;
8-isobutyl-7-methoxy-N-[4-(morpholin-4-ylmethyl)phenyl]-2-oxo-2H-chromene-3-carboxamide;
N-[4-(1-amino-1-methylethyl)phenyl]-7-ethyl-2-oxo-8-propyl-2H-chromene-3-carboxamide;
N-[4-(aminomethyl)phenyl]-7-methoxy-2-oxo-8-(2-phenylethyl)-2H-chromene-3-carboxamide;
N-[4-(aminomethyl)phenyl]-7-isopropyl-2-oxo-8-propyl-2H-chromene-3-carboxamide;
N-[4-(1-amino-1-methylethyl)phenyl]-8-ethyl-7-methoxy-2-oxo-2H-chromene-3-carboxamide;
N-[4-(2-amino-2-methylpropyl)phenyl]-7-methoxy-2-oxo-8-propyl-2H-chromene-3-carboxamide;
N-[4-(1-amino-1-methylethyl)phenyl]-7-isopropyl-2-oxo-8-propyl-2H-chromene-3-carboxamide;
N-[4-(1-aminocyclopropyl)phenyl]-8-ethyl-7-methoxy-2-oxo-2H-chromene-3-carboxamide;
N-[4-(aminomethyl)phenyl]-8-(2-hydroxyethyl)-7-methoxy-2-oxo-2H-chromene-3-carboxamide;
N-(4-{[ethyl(methyl)amino]methyl}phenyl)-7-methoxy-2-oxo-8-propyl-2H-chromene-3-carboxamide;
7-ethoxy-N-(4-{[ethyl(methyl)amino]methyl}phenyl)-2-oxo-8-propyl-2H-chromene-3-carboxamide;
N-[4-(aminomethyl)phenyl]-7-methoxy-8-(3-methylbutyl)-2-oxo-2H-chromene-3-carboxamide;
N-[4-(aminomethyl)phenyl]-8-isobutyl-7-methoxy-2-oxo-2H-chromene-3-carboxamide;
N-[4-(1-amino-1-methylethyl)phenyl]-7-methoxy-8-(3-methylbutyl)-2oxo-2H-chromene-3-carboxamide;
N-[4-(1-amino-1-methylethyl)phenyl]-8-isobutyl-7-methoxy-2-oxo-2H-chromene-3-carboxamide;
N-[4-(2-amino-1,1-dimethylethyl)phenyl]-7-methoxy-2-oxo-8-propyl-2H-chromene-3-carboxamide;
N-[4-(1-aminocyclopropyl)phenyl]-8-butyl-7-methoxy-2-oxo-2H-chromene-3-carboxamide;
N-{4-[1-(aminomethyl)cyclopropyl]phenyl}-7-methoxy-2-oxo-8-propyl-2H-chromene-3-carboxamide;
N-[4-(1-amino-1-methylethyl)phenyl]-8-butyl-7-methoxy-2-oxo-2H-chromene-3-carboxamide;
8-butyl-N-{4-[(dimethylamino)methyl]phenyl}-7-methoxy-2-oxo-2H-chromene-3-carboxamide;
8-butyl-7-methoxy-2-oxo-N-[4-(pyrrolidin-1-ylmethyl)phenyl]-2H-chromene-3-carboxamide;
N-(4-{[(3S)-3-hydroxypyrrolidin-1-yl]methyl}phenyl)-7-methoxy-2-oxo-8-propyl-2H-chromene-3-carboxamide;
7-ethoxy-N-(4-{[(3S)-3-hydroxypyrrolidin-1-yl]methyl}phenyl)-2-oxo-8-propyl-2H-chromene-3-carboxamide;
N-(4-{[(3R)-3-hydroxypyrrolidin-1-yl]methyl}phenyl)-7-methoxy-2-oxo-8-propyl-2H-chromene-3-carboxamide;
7-ethoxy-N-(4-{[(3R)-3-hydroxypyrrolidin-1-yl]methyl}phenyl)-2-oxo-8-propyl-2H-chromene-3-carboxamide;
N-[4-(1-aminocyclopropyl)phenyl]-8-isobutyl-7-methoxy-2-oxo-2H-chromene-3-carboxamide;
8-butyl-7-methoxy-N-[4-(morpholin-4-ylmethyl)phenyl]-2-oxo-2H-chromene-3-carboxamide;
N-[3-(aminomethyl)phenyl]-7-methoxy-2-oxo-8-propyl-2H-chromene-3-carboxamide;
N-[6-(aminomethyl)pyridin-3-yl]-7-methoxy-2-oxo-8-propyl-2H-chromene-3-carboxamide;
N-[5-(aminomethyl)pyridin-2-yl]-7-methoxy-2-oxo-8-propyl-2H-chromene-3-carboxamide;
7,8-dimethoxy-N-{4-[(methylamino)methyl]phenyl}-2-oxo-2H-chromene-3-carboxamide;
N-[4-(1-aminocyclopropyl)phenyl]-8-ethoxy-7-methyl-2-oxo-2H-chromene-3-carboxamide;
8-ethyl-7-methoxy-N-{4-[(1R)-1-(methylamino)ethyl]phenyl}-2-oxo-2H-chromene-3-carboxamide;
N-[4-(1-aminocyclopropyl)phenyl]-7,8-dimethoxy-2-oxo-2H-chromene-3-carboxamide;
8-ethyl-7-methoxy-N-{4-[(1S)-1-(methylamino)ethyl]phenyl}-2-oxo-2H-chromene-3-carboxamide;
N-[4-(1-amino-1-methylethyl)phenyl]-8-ethoxy-7-methyl-2-oxo-2H-chromene-3-carboxamide;
N-[4-(2-amino-1-hydroxyethyl)phenyl]-8-ethyl-7-methoxy-2-oxo-2H-chromene-3-carboxamide;
8-allyl-N-[4-(1-aminocyclopropyl)phenyl]-7-methoxy-2-oxo-2H-chromene-3-carboxamide;
8-ethyl-7-methoxy-N-{4-[1-(methylamino)cyclopropyl]phenyl}-2-oxo-2H-chromene-3-carboxamide;
8-acetyl-N-[4-(1-aminocyclopropyl)phenyl]-7-methoxy-2-oxo-2H-chromene-3-carboxamide;
N-[4-(1-aminocyclopropyl)phenyl]-8-ethoxy-7-methoxy-2-oxo-2H-chromene-3-carboxamide;
N-{3-[(dimethylamino)methyl]phenyl}-7-methoxy-2-oxo-8-propyl-2H-chromene-3-carboxamide;
N-[4-(1-aminocyclopropyl)phenyl]-7-methoxy-8-nitro-2-oxo-2H-chromene-3-carboxamide;
8-isopropoxy-7-methoxy-N-{4-[(methylamino)methyl]phenyl}-2-oxo-2H-chromene-3-carboxamide;
N-[4-(2-amino-1-hydroxyethyl)phenyl]-8-ethoxy-7-methoxy-2-oxo-2H-chromene-3-carboxamide;
N-(4-{[(2-hydroxyethyl)amino]methyl}phenyl)-7,8-dimethoxy-2-oxo-2H-chromene-3-carboxamide;
8-ethoxy-7-methoxy-N-{4-[1-(methylamino)cyclopropyl]phenyl}-2-oxo-2H-chromene-3-carboxamide;
N-(4-{[acetyl(methyl)amino]methyl}phenyl)-8-ethyl-7-methoxy-2-oxo-2H-chromene-3-carboxamide;
N-{4-[(cyclopropylamino)methyl]phenyl}-8-ethoxy-7-methoxy-2-oxo-2H-chromene-3-carboxamide;
N-{4-[(dimethylamino)methyl]-2-methoxyphenyl}-8-ethyl-7-methoxy-2-oxo-2H-chromene-3-carboxamide;
N-{4-[(dimethylamino)methyl]phenyl}-8-isopropoxy-7-methoxy-2-oxo-2H-chromene-3-carboxamide;
N-[4-(1-aminocyclopropyl)phenyl]-8-(2-hydroxyethoxy)-7-methoxy-2oxo-2H-chromene-3-carboxamide;
N-(4-{[acetyl(methyl)amino]methyl}phenyl)-7,8-dimethoxy-2-oxo-2H-chromene-3-carboxamide;
8-ethoxy-7-methoxy-N-{4-[1-methyl-1-(methylamino)ethyl]phenyl}-2-oxo-2H-chromene-3-carboxamide;
N-{4-[(2R)-2-amino-3-hydroxypropyl]phenyl}-8-isopropyl-7-methoxy-2-oxo-2H-chromene-3-carboxamide;
8-ethyl-N-(4-{[(2-hydroxyethyl)amino]methyl}-2-methylphenyl)-7-methoxy-2-oxo-2H-chromene-3-carboxamide;
8-ethoxy-N-(4-{[(2-hydroxyethyl)amino]methyl}phenyl)-7-methoxy-2-oxo-2H-chromene-3-carboxamide;
N-[4-(1-aminocyclopropyl)phenyl]-8-(cyclopropylmethoxy)-7-methoxy-2-oxo-2H-chromene-3-carboxamide;
N-{4-[(cyclopropylamino)methyl]phenyl}-8-isopropoxy-7-methoxy-2-oxo-2H-chromene-3-carboxamide;
8-ethoxy-7-methoxy-2-oxo-N-[4-(pyrrolidin-1-ylmethyl)phenyl]-2H-chromene-3-carboxamide;
N-[4-(1-aminocyclopropyl)phenyl]-8-isobutoxy-7-methoxy-2-oxo-2H-chromene-3-carboxamide;
N-[4-(1-amino-1-methylethyl)phenyl]-8-(cyclopropylmethoxy)-7-methoxy-2oxo-2H-chromene-3-carboxamide;
N-[4-(1-aminocyclopropyl)phenyl]-7-methoxy-8-(2-methoxyethoxy)-2-oxo-2H-chromene-3-carboxamide;
N-{4-[(1R)-1-acetamidoethyl]phenyl}-8-ethoxy-7-methoxy-2oxo-2H-chromene-3-carboxamide;
N-{4-[(1S)-1-acetamidoethyl]phenyl}-8-ethoxy-7-methoxy-2-oxo-2H-chromene-3-carboxamide;
N-[4-(acetamidomethyl)phenyl]-8-isopropoxy-7-methoxy-2-oxo-2H-chromene-3-carboxamide;
N-{4-[1-(dimethylamino)-1-methylethyl]phenyl}-8-ethoxy-7-methoxy-2-oxo-2H-chromene-3-carboxamide;
N-[4-(1-amino-1-methylethyl)phenyl]-8-isobutoxy-7-methoxy-2-oxo-2H-chromene-3-carboxamide;
N-[4-(1-amino-1-methylethyl)phenyl]-7-methoxy-8-{[(1S)-1-methylpropyl]oxy}-2-oxo-2H-chromene-3carboxamide;
N-[4-(1-amino-1-methylethyl)phenyl]-7-methoxy-8-{[(1R)-1-methylpropyl]oxy}-2-oxo-2H-chromene-3-carboxamide;
N-{4-[(2R)-2-amino-3-hydroxypropyl]phenyl}-8-isopropoxy-7-methoxy-2-oxo-2H-chromene-3-carboxamide;
N-{4-[(1S,2S)-2-amino-3-hydroxy-1-methoxypropyl]phenyl}-8-ethyl-7-methoxy-2-oxo-2H-chromene-3-carboxamide;
N-(4-{[(2-hydroxyethyl)amino]methyl}phenyl)-8-isopropoxy-7-methoxy-2-oxo-2H-chromene-3-carboxamide;
8-ethoxy-N-(4-{[(2-hydroxyethyl)amino]methyl}-2-methylphenyl)-7-methoxy-2-oxo-2H-chromene-3-carboxamide;
8-ethoxy-N-(4-{[(2-hydroxyethyl)(methyl)amino]methyl}phenyl)-7-methoxy-2-oxo-2H-chromene-3-carboxamide;
N-[4-(1-acetamidocyclopropyl)phenyl]-8-ethoxy-7-methoxy-2oxo-2H-chromene-3-carboxamide;
8-ethyl-7-methoxy-N-[4-(2-morpholin-4-ylethyl)phenyl]-2-oxo-2H-chromene-3-carboxamide;
7-methoxy-N-[3-(morpholin-4-ylmethyl)phenyl]-2-oxo-8-propyl-2H-chromene-3-carboxamide;
8-isopropoxy-7-methoxy-2-oxo-N-[4-(pyrrolidin-1-ylmethyl)phenyl]-2H-chromene-3-carboxamide;
8-ethoxy-7-methoxy-2-oxo-N-(4-{[(4S)-2-oxo-1,3-oxazolidin-4-yl]methyl}phenyl)-2H-chromene-3-carboxamide;
N-(4-{[acetyl(methyl)amino]methyl}phenyl)-8-isopropoxy-7-methoxy-2-oxo-2H-chromene-3-carboxamide;
8-ethoxy-7-methoxy-2-oxo-N-(4-{[(4R)-2-oxo-1,3-oxazolidin-4-yl]methyl}phenyl)-2H-chromene-3-carboxamide;
8-ethoxy-N-(4-{[(3S)-3-hydroxypyrrolidin-1-yl]methyl}phenyl)-7-methoxy-2-oxo-2H-chromene-3-carboxamide;
N-(4-{[acetyl(2-hydroxyethyl)amino]methyl}phenyl)-8-ethyl-7-methoxy-2-oxo-2H-chromene-3-carboxamide;
8-ethoxy-7-methoxy-N-[4-(morpholin-4-ylmethyl)phenyl]-2-oxo-2H-chromene-3-carboxamide;
N-{4-[(1S,2S)-2-amino-1,3-dimethoxypropyl]phenyl}-8-ethyl-7-methoxy-2-oxo-2H-chromene-3-carboxamide;
N-(4-{[(2-hydroxyethyl)amino]methyl}phenyl)-7-methoxy-8-{[(1R)-1-methylpropyl]oxy}-2-oxo-2H-chromene-3-carboxamide;
N-(4-{[(2-hydroxyethyl)amino]methyl}-2-methylphenyl)-8-isopropoxy-7-methoxy-2-oxo-2H-chromene-3-carboxamide;
N-(4-{[(2-hydroxyethyl)amino]methyl}phenyl)-7-methoxy-8-{[(1S)-1-methylpropyl]oxy}-2-oxo-2H-chromene-3-carboxamide;
8-ethoxy-N-(4-{[(2-hydroxyethyl)(methyl)amino]methyl}-2-methylphenyl)-7-methoxy-2-oxo-2H-chromene-3-carboxamide;
8-ethyl-N-(4-{[(2-hydroxyethyl)(methyl)amino]methyl}-2-methoxyphenyl)-7-methoxy-2-oxo-2H-chromene-3-carboxamide;
N-(4-{[(2-hydroxyethyl)(methyl)amino]methyl}phenyl)-8-isopropoxy-7-methoxy-2-oxo-2H-chromene-3-carboxamide;
N-[4-(1-acetamidocyclopropyl)phenyl]-8-butyl-7-methoxy-2-oxo-2H-chromene-3-carboxamide;
7-methoxy-N-[4-(2-morpholin-4-ylethyl)phenyl]-2-oxo-8-propyl-2H-chromene-3-carboxamide;
8-ethoxy-7-methoxy-N-[4-(2-morpholin-4-ylethyl)phenyl]-2-oxo-2H-chromene-3-carboxamide;
N-(4-{[(3S)-3-hydroxypyrrolidin-1-yl]methyl}phenyl)-8-isopropoxy-7-methoxy-2-oxo-2H-chromene-3-carboxamide;
N-{4-[(1S,2S)-2-(dimethylamino)-1-hydroxy-3-methoxypropyl]phenyl}-8-ethyl-7-methoxy-2-oxo-2H-chromene-3-carboxamide;
N-{4-[(1S,2S)-2-(dimethylamino)-1,3-dihydroxypropyl]phenyl}-8-ethoxy-7-methoxy-2-oxo-2H-chromene-3-carboxamide;
N-(4-{[(2-hydroxyethyl)(methyl)amino]methyl}-2-methoxyphenyl)-8-isopropoxy-7-methoxy-2-oxo-2H-chromene-3-carboxamide;
8-ethyl-N-(4-{[(2-hydroxyethyl)(methyl)amino]methyl}-2-methylphenyl)-7-methoxy-2-oxo-2H-chromene-3-carboxamide;
N-{4-[(1S)-1-aminoethyl]phenyl}-8-ethoxy-7-methoxy-2-oxo-2H-chromene-3-carboxamide;
N-[4-(1-aminocyclopentyl)phenyl]-8-ethyl-7-methoxy-2-oxo-2H-chromene-3-carboxamide;
N-[4-(1-aminocyclopropyl)phenyl]-7-ethoxy-2-oxo-2H-chromene-3-carboxamide;
8-allyl-N-[4-(1-amino-1-methylethyl)phenyl]-6methoxy-2-oxo-2H-chromene-3-carboxamide;
N-{4-[(1R)-1-aminoethyl]phenyl}-8-ethoxy-7-methoxy-2-oxo-2H-chromene-3-carboxamide;
N-{4-[(1R)-1-aminopropyl]phenyl}-8-ethoxy-7-methoxy-2-oxo-2H-chromene-3-carboxamide;
N-{4-[(1S)-1-aminobutyl]phenyl}-8-ethoxy-7-methoxy-2-oxo-2H-chromene-3-carboxamide;
N-{4-[(1R)-1-aminobutyl]phenyl}-8-ethoxy-7-methoxy-2-oxo-2H-chromene-3-carboxamide;
N-[4-(1-amino-1-methylethyl)phenyl]-7,8-dimethoxy-2-oxo-2H-chromene-3-carboxamide;
8-ally-N-[4-(1-amino-1-methylethyl)phenyl]-7-methoxy-2-oxo-2H-chromene-3-carboxamide;
N-[4-(1-amino-1-methylethyl)phenyl]-8-ethoxy-7-methoxy-2-oxo-2H-chromene-3-carboxamide;
N-[4-(1-amino-1-methylethyl)phenyl]-7-methoxy-8-(2-methoxyethoxy)-2-oxo-2H-chromene-3-carboxamide;
N-[4-(1-amino-1-methylethyl)phenyl]-2-oxo-7,8,9,10-tetrahydro-2H-benzo[h]chromene-3-carboxamide;
N-[4-(aminomethyl)phenyl]-2-oxo-7,8,9,10-tetrahydro-2H-benzo[h]chromene-3-carboxamide;
7-methoxy-2-oxo-8-propyl-N-(1,2,3,4-tetrahydroisoquinolin-6-yl)-2H-chromene-3-carboxamide;
N-(1-imino-2,3-dihydro-1H-isoindol-5-yl)-7-methoxy-2-oxo-8-propyl-2H-chromene-3carboxamide;
N-(4-{[(8-allyl-7-methoxy-2-oxo-2H-chromen-3-yl)carbonyl]amino}benzoyl)glycine;
N-[4-(1-aminocyclopropyl)phenyl]-8-tert-butyl-2-oxo-2H-chromene-3-carboxamide;
N-[4-(1-amino-1-methylethyl)phenyl]-8-tert-butyl-2-oxo-2H-chromene-3-carboxamide;
N-[4-(1-aminocyclopropyl)phenyl]-8-ethoxy-2-oxo-2H-chromene-3-carboxamide;
8-allyl-N-[4-(1-aminocyclopropyl)phenyl]-2-oxo-2H-chromene-3-carboxamide;
8-allyl-N-(4-{[(2-hydroxyethyl)amino]methyl}phenyl)-2-oxo-2H-chromene-3-carboxamide; or a physiologically hydrolysable derivative thereof, a salt, hydrate and/or solvate thereof.

6. A compound according to claim 1, in free form or in a pharmaceutically acceptable salt form, for use as a pharmaceutical.

7. A pharmaceutical composition comprising a compound according to claim 1, in free form or in pharmaceutically acceptable salt form, in association with a pharmaceutically acceptable diluent or carrier therefore.

8. A pharmaceutical combination comprising a compound according to claim 1, in free form or in a pharmaceutically acceptable salt form, and a further agent selected from immunosuppressant, immunomodulatory, anti-inflammatory, chemotherapeutic and anti-infectious agents.

9. A process for the production of the compound of formula (I) according to claim 1, which process comprises
either reacting a compound of formula (II), 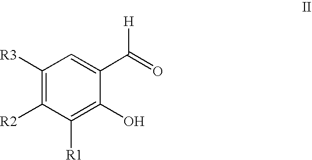
wherein R₁, R₂, and R₃ are as defined in claim 1,
with a compound of formula (III), 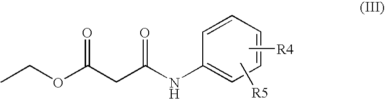
wherein R₄ and R₅ are as defined in claim 1;
or reacting a compound of formula (V), 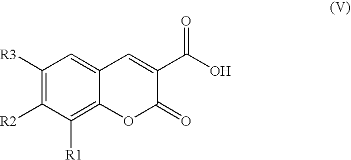
wherein R₁, R₂ and R₃ are as defined in claim 1,
with a compound of formula (VI), 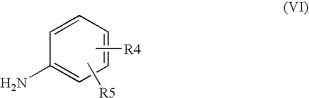
wherein R₄ and R₅ are as defined in claim 1;
with the proviso as defined in claim 1;
and, where required, converting the resulting compound of formula (I) obtained in free form to a salt form or vice versa, as appropriate.

10. A method for treating or preventing disorders or diseases mediated by T lymphocytes, in a subject in need of such treatment, which method comprises administering to said subject an effective amount of a compound according to claim 1, or a pharmaceutically acceptable salt thereof.
